(19) United States
(12) Patent Application Publication
(10) Pub. No.: US 2026/0076734 A1
(43) Pub. Date: Mar. 19, 2026

(54) Method and Device for Whole Organ Pulsed Electric Field Application for Irreversible Electroporation

(71) Applicant: University of Massachusetts, Westborough, MA (US)

(72) Inventors: Govind Srimathveeravalli, Westborough, MA (US); Mary Chase Sheehan, Westborough, MA (US); Shengwei Wu, Westborough, MA (US)

(21) Appl. No.: 19/328,944

(22) Filed: Sep. 15, 2025

Related U.S. Application Data
(60) Provisional application No. 63/694,232, filed on Sep. 13, 2024.

(51) Int. Cl. A61B 18/14; A61B 18/00; A61K 33/06; A61K 33/243; A61K 38/14; A61N 1/32

(52) U.S. Cl. CPC A61B 18/1492; A61K 33/06; A61K 33/243; A61K 38/14; A61N 1/327; A61B 2018/00023; A61B 2018/00077; A61B 2018/0022; A61B 2018/00267; A61B 2018/00517; A61B 2018/00577; A61B 2018/00613; A61B 2018/1467

(57) ABSTRACT

An exemplary pulsed electric field system and method that can uniformly emit electrical pulses via use of a conductivity-controlled liquid medium to penetrate a pre-defined depth of a target tissue for a whole or region of a hollow or tubular organ to induce a tissue response. The conductivity-controlled liquid medium allows more uniform electrical pulses to be generated with less localized or focal bursts of pulses, which can reduce the required power rating of the electric generator for the pulses. The pulsed electric field system and method can be used for irreversible electroporation and subsequent exfoliation of the surface tissue, electroporation, pulsed field ablation, or cell stimulation.

### CROSS-REFERENCE TO RELATED APPLICATIONS

This US application claims priority to, and the benefit of, U.S. Provisional Patent Application No. 63/694,232, filed Sep. 13, 2024, entitled “METHOD AND DEVICE FOR WHOLE BLADDER PULSED ELECTRIC FIELD APPLICATION FOR CANCER TREATMENT,” which is incorporated by reference herein in its entirety.

### STATEMENT REGARDING FEDERALLY SPONSORED RESEARCH OR DEVELOPMENT

This invention was made with government support under Grant #R01DK129990 awarded by the National Institutes for Health and Grant #2338949 awarded by the National Science Foundation. The government has certain rights in the invention.

### BACKGROUND

The molecular and genetic characteristics of bladder cancer render it likely to develop resistance and escape therapies that targets specific or even multiple biological pathways. The surgical removal of all bladder urothelium during radical cystectomy can provide durable cancer control, but its use is often restricted to high-risk patients with muscle-invasive bladder cancer or after failure of intravesical therapy. Research has demonstrated that ablation can rapidly eliminate all urothelium from the bladder, similar to radical cystectomy, and is minimally invasive, bladder-sparing.

There is a benefit to improving the elimination of urothelium via ablation, and other epithelial tissue, and like minimally invasive systems and techniques.

### SUMMARY

An exemplary pulsed electric field system and method are provided that can uniformly emit electrical pulses via use of a conductivity-controlled liquid medium to penetrate a pre-defined depth of a target tissue for a whole or region of a hollow or tubular organ to induce a tissue response (e.g., irreversible electroporation and subsequent exfoliation of the surface tissue, electroporation, pulsed field ablation, cell stimulation). Electroporation employs brief electrical pulses to temporarily increase the permeability of cell membranes, allowing the introduction of molecules (such as DNA or drugs) into cells. Irreversible electroporation and pulsed field ablation employ microsecond to short millisecond pulses to induce a permanent change in the cell membrane as a type of ablation. Whereas irreversible electroporation is characterized as a treatment for any tissue type or organ, pulsed field ablation is specific to cardiac cells or tissue. Cell stimulation via electrical stimulation can trigger a cellular response to induce a change in cell activity, e.g., proliferation, cytokine production, differentiation, etc. The conductivity-controlled liquid medium allows more uniform electrical pulses to be generated with fewer localized or focal bursts of pulses, which can reduce the required power rating of the electric generator for the pulses. The irreversible electroporation/ablation can rapidly eliminate all urothelium from the bladder, similar to radical cystectomy, and in a non-invasive manner. The exemplary pulsed electric field system and method can be employed for the bladder, stomach, small intestine, large intestine, blood vessels, fallopian tubes, urethra, heart, and other hollow or tubular organs, as well as ducts typically filled with fluid.

The exemplary pulsed electric field device may be optimized for clinical usage with a conductivity-controlled liquid medium to deposit energy in the entire circumference, surface, or region of the organ into a predictable depth or tissue layer. The controllability of the deposition of energy facilitates and can be used in combination with a variety of treatments (therapeutic, gene, cell impermeable agent, or other biological intervention therapy described or referenced herein).

The control of depth, particularly the superficial layer, and the application across a large or whole area of the organ, provide a new approach for cancer treatment. In particular, for the bladder, the exemplary pulsed electric field system and method can be used to deposit electrical energy across the whole bladder to exfoliate the entire or desired portion of the urothelial layer, to remove much of the surface cells or tissue to which the cancers come, and/or reset the entire bladder urothelial layer. The exemplary pulsed electric field system and method can be used in combination with cell cell-impermeable agent, e.g., calcium, or others described herein, to promote exfoliation or cell death of surface cells and tissue.

The exemplary pulsed electric field system and method can be tailored to selectively target a response in certain cells (e.g., cancer cells) while not affecting other cells (e.g., T-cells, macrophages, etc.).

The exemplary pulsed electrical field device may include features to address technical challenges associated with large volume surface energy deposition. To deliver electrical energy over a large volume surface, many electrodes are often desired. Increasing the number of electrodes facilitates the distribution of energy to the tissue, but at a tradeoff of other technical issues, e.g., device complexity, uneven electrical distribution that can lead to localized heating. The conductivity-controlled liquid medium mediates the electrical energy emissions and the nearby or contacting tissue to lessen or reduce focalized electrical emission and, thus, associated focalized thermal effects, reduce the need to have a large number of electrodes, provide a medium for cooling/thermal regulation, and provide a medium for secondary or tertiary control via therapeutic, gene, or other biological intervention therapy. The exemplary pulsed electric field system and method can operate without reliance on thermal effects. The exemplary pulsed electric field system and method can operate in combination with thermal effects.

In addition to treatments, e.g., cancer treatments, the exemplary pulsed electrical field device can be alternatively used for liquid biopsy, cell stimulation, and electroporation, among others.

In an aspect, a system is disclosed comprising an expandable electrode catheter comprising: a tubular member having a first end and a second end; and a plurality of electrodes comprising at least 4 electrodes including a first electrode and a second electrode, the plurality of electrodes housed in the first end of the tubular member and configured (i) to be stowed within the tubular member in a first configuration and (ii) to extend out of the tubular member, via movement of the tubular member or movement of the plurality of electrodes, to be in a second configuration, wherein during deployment the first electrode extends in a first angular direction and second electrode extends in a second angular direction opposite to the first angular direction so each come into proximity or contact with a surface tissue of a hollow or tubular organ to fill a shape of the hollow or tubular organ, wherein each of the plurality of electrodes is configured to uniformly directly or indirectly emit a plurality of electrical pulses in a liquid medium (e.g., while in contact with the tissue or only through the liquid medium) to penetrate at least a pre-defined depth into the surface tissue as a pulsed electric field application to induce a tissue response (e.g., irreversible electroporation and subsequent exfoliation, electroporation, pulsed field ablation, cell stimulation, and other non-thermal ablation) of at least the surface tissue of the hollow or tubular organ.

In some embodiments, the flexible tubular member comprises a channel member that extends, at least, between the first end and the second end of the tubular member for introduction of a liquid, as a portion of the liquid medium, to be dispensed during the pulsed electric field application by the expandable electrode catheter (e.g., wherein the liquid and liquid medium can limit the focal or localized ablation of the surface tissue of the hollow or tubular organ or the off-target destruction of other biological material in the hollow or tubular organ, such as blood or other bodily fluids).

In some embodiments, the expandable electrode catheter further comprises an expandable sheath (e.g. balloon) comprising a non-conductive material, the expandable sheath housed in the first end of the tubular member and configured (i) to be stowed within the tubular member in a stowed configuration and (ii) to extend out of the tubular member to define a sheath volume defined within the plurality of electrodes, wherein the sheath volume when deployed reduces a volume in a space in the hollow or tubular organ for delivery of electrical pulse in the liquid medium to the hollow or tubular organ.

In some embodiments, the expandable electrode catheter comprises a tubular sheath forming in part the tubular member, the tubular sheath having a first end and a second end; and a shaft housed within the tubular sheath and configured to be displaced (e.g., slidable) within the sheath, the shaft having a first end and a second end, wherein the second end of the shaft is configured to controllably move to the second end of the sheath to induce deployment of the plurality of electrodes.

In some embodiments, the expandable electrode catheter comprises a tubular sheath forming in part the tubular member, the tubular sheath having a first end and a second end; and a shaft housed within the tubular sheath, a portion of the tubular sheath being configured to be displaced (e.g., slidable) with respect to the shaft, the shaft having a first end and a second end, wherein the second end of the shaft is configured to controllably move to the second end of the sheath to induce deployment of the plurality of electrodes.

In some embodiments, the plurality of electrodes form a basket configured to conform to a shape of the hollow or tubular organ.

In some embodiments, the plurality of electrodes are between 4 and 100 electrode members, wherein the plurality of electrodes are configured to flexibly extend to contact and oppose a lumen wall as the surface tissue without losing contact.

In some embodiments, the electrode members are configured to extend about 1 mm-75 mm from the tubular member to have at least one electrode member positionable 0 to 5 mm from a lumen wall as the tissue surface.

In some embodiments, the expandable electrode catheter further comprises an expandable non-conductive envelop, the expandable non-conductive envelope having a plurality of expandable members housed at the first end of the tubular member and configured (i) to be stowed to define an outer surface the tubular member in a stowed configuration and (ii) to extend or expand from the tubular member to define an external envelope volume, wherein the plurality of electrodes expands in the second configuration to be defined within the external envelope volume, wherein the expandable non-conductive envelope defines a pre-defined gap or distance between the plurality of electrodes and the surface tissue.

In some embodiments, the expandable electrode catheter is coupled to a handle, the handle being manipulatable to move the plurality of electrodes within the hollow or tubular organ.

In some embodiments, the system further includes an electrical instrument configured to deliver the plurality of electrical pulses to the plurality of electrodes.

In some embodiments, the electrical instrument is configured, via a control program for a pulsed electric field application (e.g., electroporation, irreversible electroporation, pulsed field ablation, or targeted cell stimulation), to generate a monophasic electro pulse having a square wave.

In some embodiments, the electrical instrument is configured, via a control program for irreversible electroporation, to output a voltage range of 50 V-1500V (e.g., 50 Vₚₑₐₖ-1500 Vₚₑₐₖ), and a pulse width of 10 μs-1000 μs (e.g., 50-150 μs).

In some embodiments, the electrical instrument is configured, via a control program for irreversible electroporation, to generate electropulses having a selectable interpulse delay (e.g., triggerable via an external biosignal, e.g., muscle contraction, nerve excitation, or cardiac rhythm).

In some embodiments, the electrical instrument is configured, via a control program for irreversible electroporation, to deliver a pre-defined number of electropulses (e.g., having a minimum of 3-8 electropulses and a maximum of 10,000 electropulses).

In some embodiments, the electrical instrument is configured, via a control program for irreversible electroporation, to generate biphasic electropulses comprising symmetric or asymmetric square waves.

In some embodiments, the electrical instrument is configured, via the control program, to generate the biphasic electropulses at a voltage range of 100 V-1500 V (e.g., 100Vₚₚ-1500 Vₚₚ), and a pulse width of 5 ns-50 μs.

In some embodiments, the electrical instrument is configured, via the control program, to generate electropulses comprising a 5 ns-1 s interpulse delay.

In some embodiments, the electrical instrument is configured, via the control program, to deliver a pre-defined number of electropulses (e.g., having a minimum of 3-8 electropulses and a maximum of 10,000 electropulses).

In some embodiments, the system further includes a liquid introducer device, the liquid introducer device being configured, via control, to introduce the liquid into the hollow or tubular organ during the pulsed electric field application and maintain the conductivity ratio in a pre-defined range (e.g., a conductivity ratio between 1 and 10 between the liquid medium in the hollow or tubular organ and the surface tissue of the hollow or tubular organ when the plurality of electrodes are intended to contact the surface tissue during the delivery of the electropulses) (e.g., a conductivity ratio between 0.1 and 0.5 between the liquid medium in the hollow or tubular organ and the surface tissue of the hollow or tubular organ when the plurality of electrodes are intended to not contact the surface tissue during the delivery of the electropulses (e.g., when plastic spacer cage is employed)).

In some embodiments, the system further includes a recirculation loop instrument, the recirculation loop instrument being coupled to the expandable electrode catheter or a delivery device to actively recirculate the liquid and portion of the liquid medium during the pulsed electric field application (e.g., to reduce localized tissue heating).

In another aspect, an expandable electrode catheter is disclosed comprising a tubular member having a first end and a second end; and a plurality of electrodes comprising at least 4 electrodes including a first electrode and a second electrode, the plurality of electrodes housed in the first end of the tubular member and configured (i) to be stowed within the tubular member in a first configuration and (ii) to extend out of the tubular member, via movement of the tubular member or movement of the plurality of electrodes, to be in a second configuration, wherein during deployment the first electrode extends in a first angular direction and second electrode extends in a second angular direction opposite to the first angular direction so each come into proximity or contact with a surface tissue of a hollow or tubular organ to fill a shape of the hollow or tubular organ, wherein each of the plurality of electrodes is configured to uniformly emit a plurality of electrical pulses in a liquid medium to penetrate at least a pre-defined depth into the surface tissue to induce irreversible electroporation and subsequent exfoliation of the surface tissue of the hollow or tubular organ.

In another aspect, a method of treating bladder cancer is disclosed, the method includes introducing a plurality of electrodes of an expandable electrode catheter (e.g., for any of the above discussed catheter) into a hollow or tubular organ of a patient (e.g., human or animal patient); introducing or adjusting conductivity of a liquid medium in the hollow or tubular organ; and uniformly delivering, for the conductivity of the liquid medium, an effective amount of electropulses to the surface tissue or targeted tissue of the hollow or tubular organ to penetrate at least a pre-defined depth into the surface tissue or the targeted tissue to induce a tissue response (e.g., irreversible electroporation and subsequent exfoliation, electroporation, pulsed field ablation, cell stimulation, and other non-thermal ablation) of at least the surface tissue of the hollow or tubular organ.

In some embodiments, the method includes introducing a conductive fluid (e.g., an iso-osmolar conductive fluid, or biological fluid, e.g., urine, etc.) into the hollow or tubular organ prior to delivering the electropulses.

In some embodiments, the method includes introducing the conductive fluid (e.g., the iso-osmolar conductive fluid, or biological fluid, e.g., urine, etc.) into the hollow or tubular organ during the delivery of the electropulses.

In some embodiments, the introduced conductive fluid is added to generate a conductivity ratio between 1 and 10 between the liquid medium in the hollow or tubular organ and the surface tissue of the hollow or tubular organ (e.g., when the plurality of electrodes are intended to contact the surface tissue during the delivery of the electropulses.

In some embodiments, the introduced conductive fluid is added to generate a conductivity ratio between 0.1 and 0.5 between the liquid medium in the hollow or tubular organ and the surface tissue of the hollow or tubular organ (e.g., when the plurality of electrodes are intended to not contact the surface tissue during the delivery of the electropulses (e.g., when plastic spacer cage is employed).

In some embodiments, the method includes measuring, via the expandable electrode catheter, the conductivity of the liquid medium in the hollow or tubular organ prior to or during the delivery of the electropulses; and determining (e.g., via software) a volume of conductive fluid to introduce into the hollow or tubular organ to adjust the conductivity of the liquid medium in the hollow or tubular organ to a pre-defined desired conductivity ratio or conductivity ratio range between the liquid medium in the hollow or tubular organ and the surface tissue of the hollow or tubular organ.

In some embodiments, the method includes introducing a cell impermeable agent prior to or during the delivery of the electropulses to increase cytotoxicity in the hollow or tubular organ.

In some embodiments, the cell impermeable agent is selected from the group consisting of calcium (e.g., Ca2+), bleomycin, cisplatin, or a combination thereof.

In some embodiments, the partial or full epithelial or urothelial tissue exfoliation is used to treat cancer.

In some embodiments, the partial or full epithelial or urothelial tissue exfoliation is used to treat bladder cancer, wherein pulse configurations are selectable to selectively induce a response in the epithelial or cancer cells and a lesser response to T cells (e.g., sparing one cell type, e.g., T cells, while killing epithelial or cancer cells).

In some embodiments, the partial or full epithelial or urothelial tissue exfoliation is to promote or induce deposition and activity of immune checkpoint inhibitors targeting PD1/PD-L1, TIM/LAG, and CTLA at the hollow or tubular organ.

In some embodiments, the partial or full epithelial or urothelial tissue exfoliation is to promote or induce deposition of therapeutics (e.g., cisplatin, gemcitabine, mitomycin, and other platinum drugs) at the hollow or tubular organ.

In some embodiments, the partial or full epithelial or urothelial tissue exfoliation is to promote or induce the delivery of oncolytic virus, peptides, cytokines, or a combination thereof at the hollow or tubular organ.

In some embodiments, the partial or full epithelial or urothelial tissue exfoliation is to promote or induce the delivery of cell therapy products such as TCR or CAR-engineered T cells, NK cells, and macrophages, to the hollow or tubular organ.

In some embodiments, the partial or full epithelial or urothelial tissue exfoliation is to promote or induce gene transfer by delivery of mRNA, linear, circular DNA, plasmid, or a combination thereof at the hollow or tubular organ.

In some embodiments, the method includes deploying an expandable sheath (e.g. balloon) of the expandable electrode catheter, the expandable sheath comprising a non-conductive material and being housed in the first end of the tubular member and configured (i) to be stowed within the tubular member in a stowed configuration and (ii) to extend out of the tubular member to define a sheath volume defined within the plurality of electrodes, wherein the sheath volume is deployed to reduce a volume in a space in the hollow or tubular organ for delivery of electrical pulse in the liquid medium to the hollow or tubular organ.

In some embodiments, the method includes deploying an expandable non-conductive envelope of the expandable electrode catheter, the expandable non-conductive envelope having a plurality of expandable members housed at the first end of the tubular member and configured (i) to be stowed to define an outer surface the tubular member in a stowed configuration and (ii) to extend or expand from the tubular member to define an external envelope volume, wherein the plurality of electrodes expands to be defined within the external envelope volume, wherein the expandable non-conductive envelope defines a pre-defined gap or distance between the plurality of electrodes and the surface tissue.

In some embodiments, the method includes imaging the expandable electrode catheter for guided delivery of the electrical pulses in the liquid medium.

In some embodiments, the method includes adjusting delivery of the electropulses, wherein the adjustment configures the electropulses as monophasic electropulses comprising symmetric or asymmetric square waves having: (i) a voltage range of 50 V-1500V (e.g., 50 Vₚₑₐₖ-1500 Vₚₑₐₖ), and pulse width of 10 μs-1000 μs (e.g., 50-150 μs), (ii) an interpulse delay (e.g., triggerable via an external biosignal, e.g., muscle contraction, nerve excitation, or cardiac rhythm), and/or (iii) pre-defined number of electropulses (e.g. having a minimum of 3-8 electropulses and a maximum of 10,000 electropulses), wherein the parameters being definable by a control program for irreversible electroporation.

In some embodiments, the method includes adjusting delivery of the electropulses, wherein the adjustment configures the electropulses as biphasic electropulses comprising symmetric or asymmetric square waves having: (i) a voltage range of 100 V-1500 V (e.g., 100Vₚₚ-1500 Vₚₚ), and pulse width of 5 ns-50 μs, (ii) an interpulse delay (e.g., triggerable via an external biosignal, e.g., muscle contraction, nerve excitation, or cardiac rhythm, e.g., having an interpulse delay of 5 ns-1 s), and/or (iii) pre-defined number of electropulses (e.g. having a minimum of 3-8 electropulses and a maximum of 10,000 electropulses), wherein the parameters being definable by a control program for irreversible electroporation.

### BRIEF DESCRIPTION OF THE DRAWINGS

These and other features, aspects, and advantages of the present invention will become better understood with reference to the following description, appended claims, and accompanying drawings.

FIGS. 1A-1E show an example pulsed electric field system that can uniformly emit electrical pulses via the use of an expandable electrode catheter and a conductivity-controlled liquid medium to penetrate a pre-defined depth of a target tissue for a whole or region of a hollow or tubular organ in accordance with an illustrative embodiment.

FIGS. 2A-2C show example instrumentation implementation and electrode configurations for the pulsed electric field system of FIG. 1A-1E in accordance with an illustrative embodiment.

FIGS. 3A-3F show different mechanical configurations for the pulsed electric field device of FIGS. 1A-1E in accordance with various embodiments.

FIGS. 4A-4J show example method 400 for the pulsed electric field application in accordance with various embodiments.

FIGS. 5A-5I shows a pulsed electric field methodology and computation simulation results of modeling to design a pulsed electric field device.

FIGS. 6A-6E show experimental results for pulsed electric field delivery in tumor mimics to assess irreversible electroporation ablation regions.

FIGS. 7A-7I show experimental results for calcium-assisted irreversible electroporation (CAIR) with enhanced electric field strength uniformity.

FIG. 8A-8C show experimental results for electroporation and drug delivery in the whole bladder or whole organ.

FIG. 9 shows experimental results for liquid biopsy generation.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference was individually incorporated by reference.

### Definition

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term “treatment” as used herein can include any treatment of a disease disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Example System

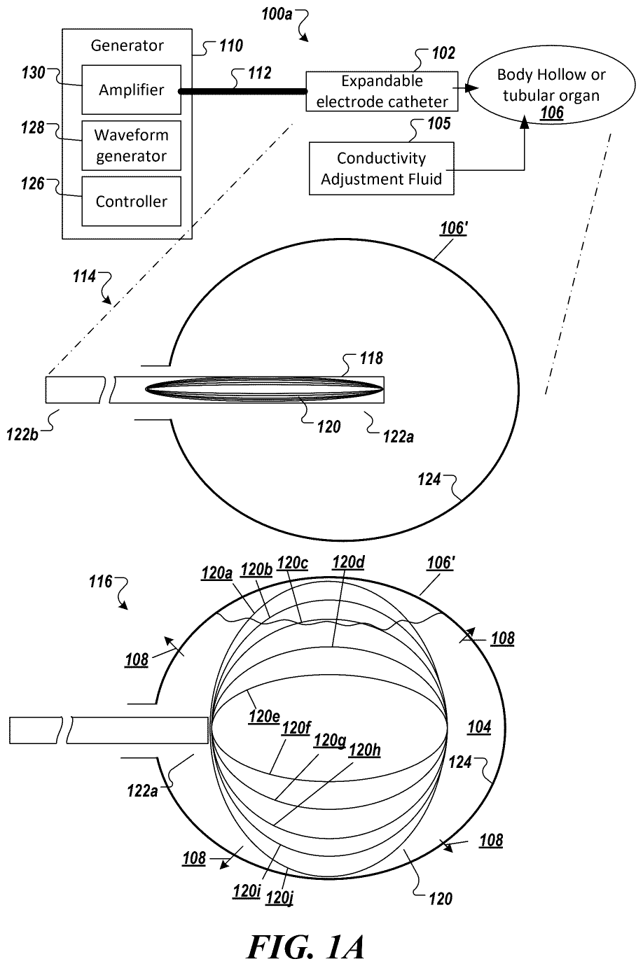

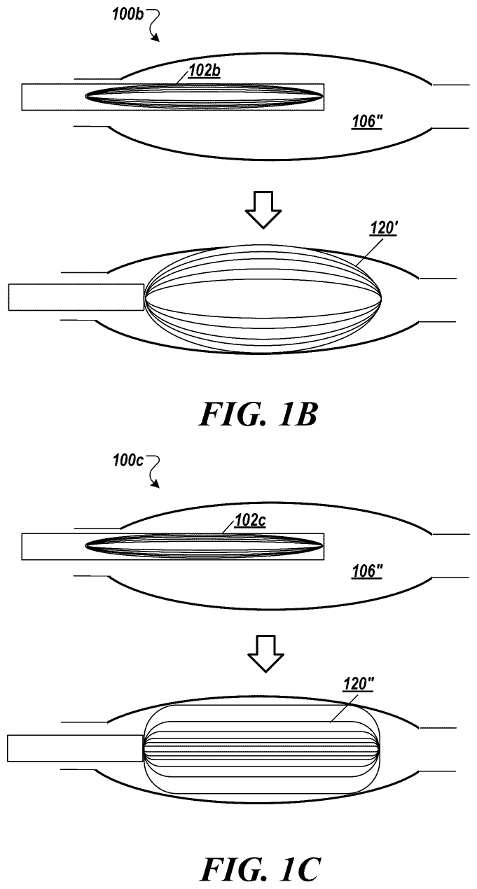

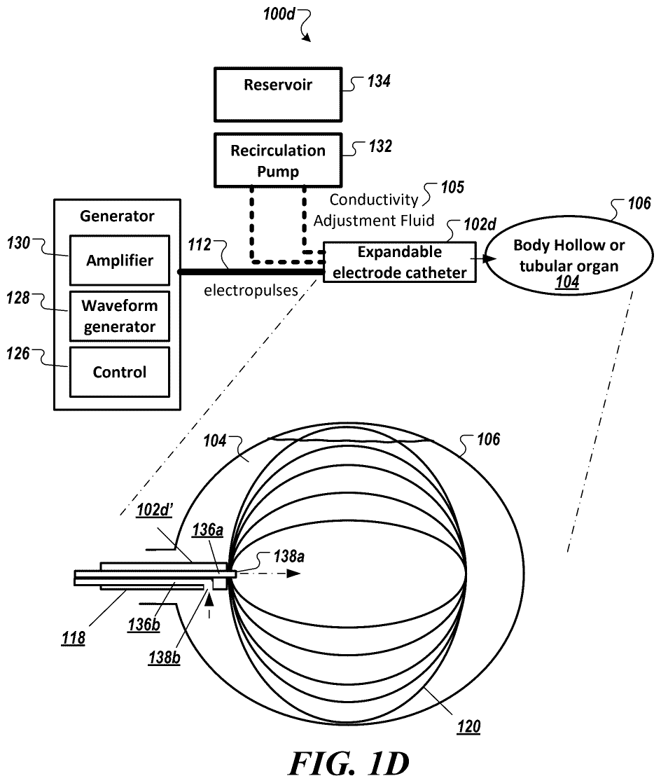

FIG. 1A shows an example pulsed electric field system 100 (shown as 100a, 100b, 100c, 100d, 100e) that can uniformly emit electrical pulses via use of an expandable electrode catheter 102 and a conductivity-controlled liquid medium 104 to penetrate a pre-defined depth of a target tissue for a whole or region of a hollow or tubular organ 106 in accordance with an illustrative embodiment. The pulsed electric field 108 induces a tissue response to invoke, depending on the electrical characteristics, irreversible electroporation and subsequent exfoliation of the surface tissue, electroporation, pulsed field ablation, and cell stimulation, among others. Electroporation employs brief electrical pulses to temporarily increase the permeability of cell membranes, allowing the introduction of molecules (such as DNA or drugs) into cells. Irreversible electroporation and pulsed field ablation employ microsecond to short millisecond pulses to induce a permanent change in the cell membrane as a type of ablation. Whereas irreversible electroporation is characterized as a treatment for any tissue type or organ, pulsed field ablation is specific to cardiac cells or tissue. Cell stimulation via electrical stimulation can trigger a cellular response to induce a change in cell activity, e.g., proliferation, cytokine production, differentiation, etc.

The conductivity-controlled liquid medium 104 allows more uniform electrical pulses 108 to be generated with less localized or focal bursts of pulses, which can reduce the required power rating of the electric generator for the pulses. The irreversible electroporation/ablation can rapidly eliminate all urothelium from the bladder, similar to radical cystectomy, and in a non-invasive mechanical manner. The pulsed electric field device can be surgical inserted for certain applications or when desired.

In the example shown in FIG. 1A, the expandable electrode catheter 102 operates with a generator 110 through a cable 112. The expandable electrode catheter 102 is configured to be inserted into the hollow or tubular organ 106 (shown as 106′) while in the stowed configuration 114 and then deployed in the hollow or tubular organ 106′ to the deployed configuration 116.

The expandable electrode catheter 102 includes a tubular member 118 and a plurality of electrodes 120. The tubular member 118 has a first end 122a and a second end 122b. The plurality of electrodes 120 includes at least 4 electrodes (e.g., shown as 120a, 120b, 120c, . . . 120j) that is housed in the first end 122a of the tubular member 118 when in the deployed configuration 114 and is configured to extend out of the tubular member 118, via movement of the tubular member or movement of the plurality of electrodes, to be in the deployed configuration 116. During deployment, the electrodes 120 extend in different angular directions to each other to come into proximity or contact with the surface tissue 124 of the hollow or tubular organ 106, e.g., to fill the shape of the hollow or tubular organ 106. The conductivity-controlled liquid medium 104 is a natural or synthetic liquid introduced into the hollow or tubular organ 106.

Conductivity-controlled liquid medium and adjustments. The conductivity-controlled liquid medium 104 has a defined conductance or resistance property and can be adjusted to a conductivity ratio by introduction of a conductivity adjustment fluid 105. The conductivity ratio of the conductivity-controlled liquid medium 104 may be established (i) between 1 and 10 between the liquid medium 104 in the hollow or tubular organ 106 and the surface tissue 124 when the electrodes 120 are intended to contact the target tissue (e.g., surface tissue) during the delivery of the electropulses and (ii) between 0.1 and 0.5 when the electrodes 120 are intended to not contact the target tissue. The conductivity-controlled liquid medium 104 may include calcium, bleomycin, cisplatin, or other cell impermeable agent introduced by the conductivity adjustment fluid 105. In some embodiments, the conductivity-controlled liquid medium 104 has an established conductivity ratio (for contact pulsed electric field application with a contact target tissue) between a value of 1-2, 2-3, 3-4, 4-5, 5-6, 6-7, 7-8, 8-9, 9-10, 1-3, 2-4, 3-5, 4-6, 5-7, 6-8, 7-9, 8-10. In some embodiments, the conductivity-controlled liquid medium 104 has an established conductivity ratio (for contactless pulsed electric field application with the nearby tissue) between a value of 0.1-0.2, 0.2-0.3, 0.3-0.4, 0.4-0.5, 0.1-0.3, 0.2-0.4, 0.3-0.5.

Table 1 shows example conductivity ratios to be employed for the pulsed electric field operation.

TABLE 1

| | |
|---|---|
| Contact | Conductivity ratio between 1 and 10 between the |
| | liquid medium in the hollow or tubular organ and |
| | the target tissue (e.g., surface tissue) of the |
| | hollow or tubular organ |
| Non-Contact | Conductivity ratio between 0.1 and 0.5 between the |
| | liquid medium in the hollow or tubular organ and |
| | the target tissue (e.g., surface tissue) of the |
| | hollow or tubular organ |

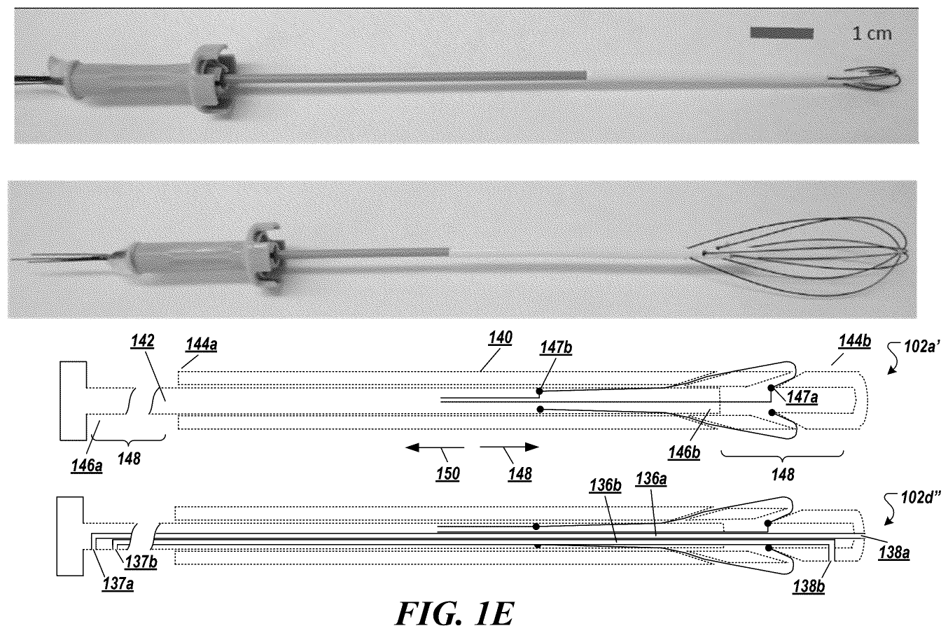

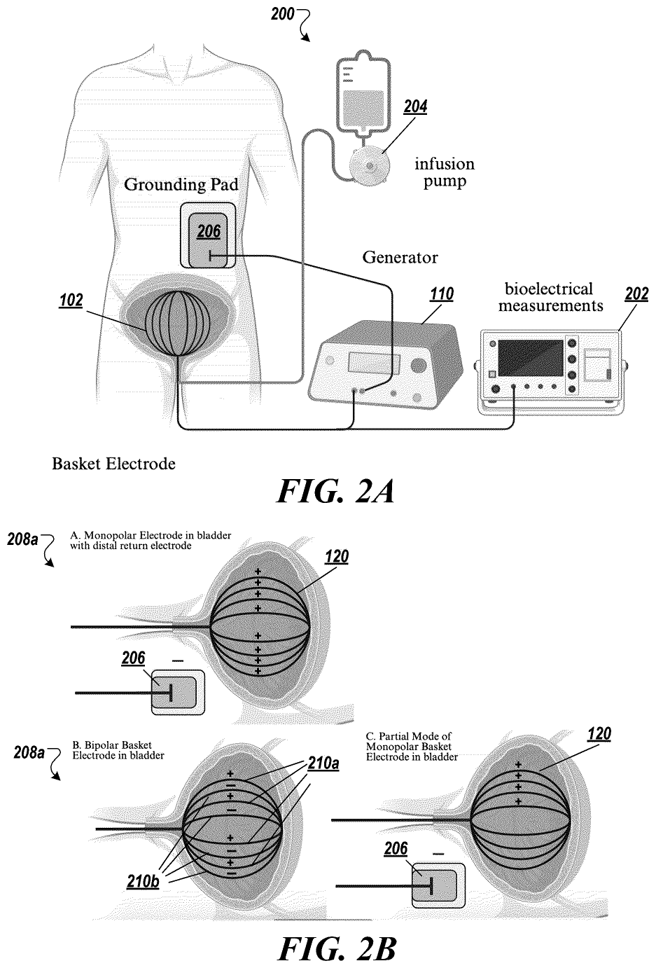

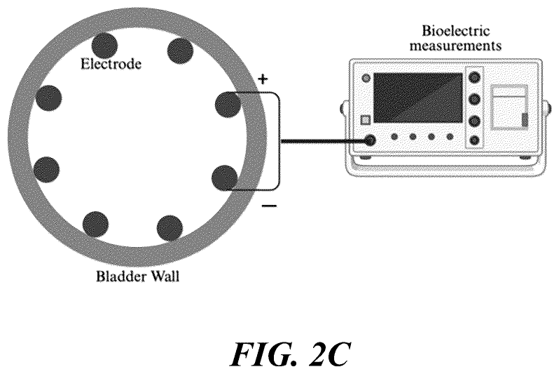

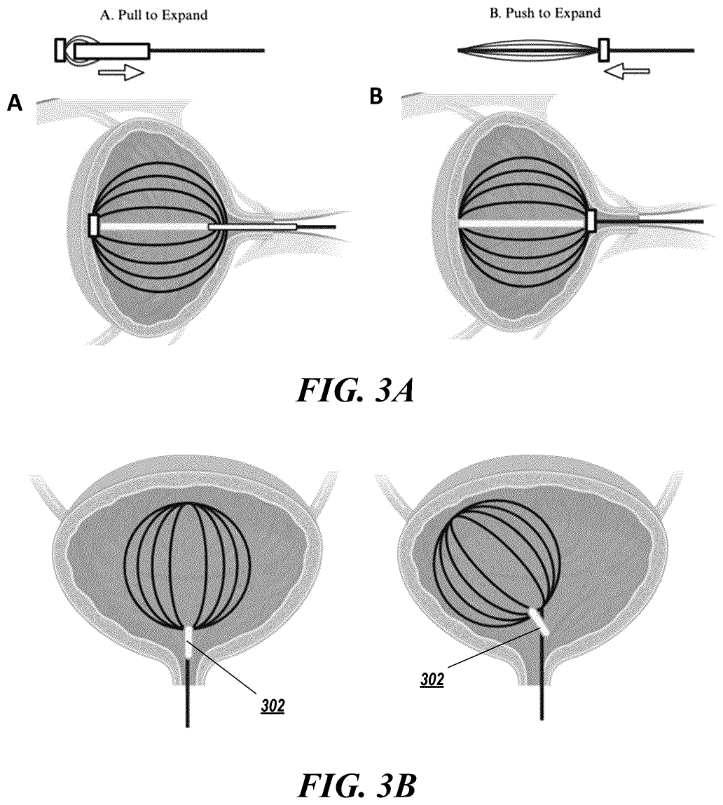

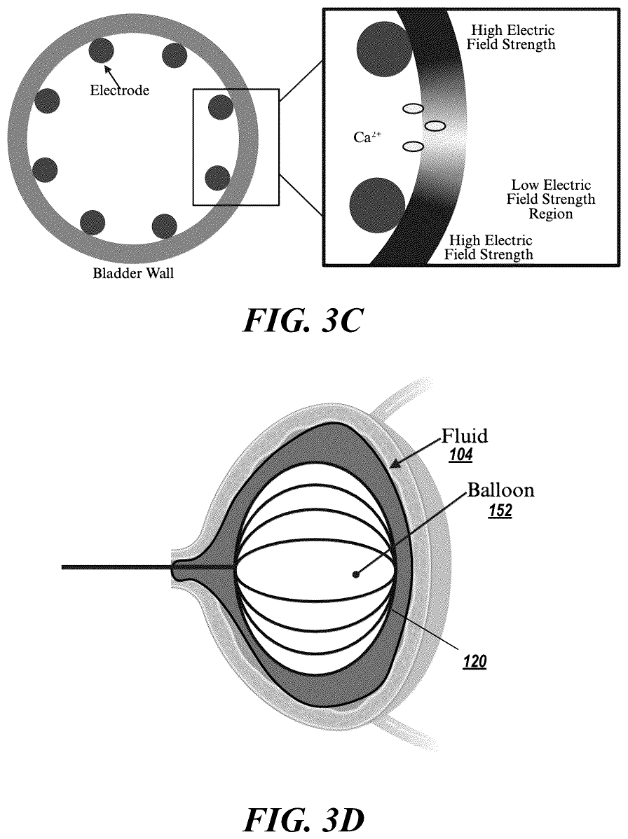

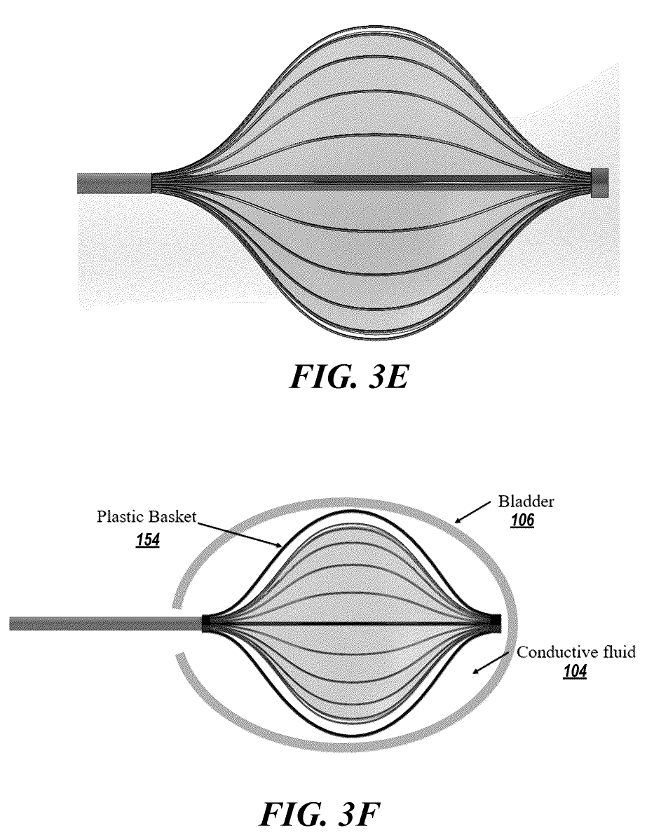

Electrodes. The electrodes 120 are configured to uniformly, directly or indirectly, emit the plurality of electrical pulses 108 in the liquid medium 104 (e.g., while in contact with the tissue or only through the liquid medium) to penetrate at least a pre-defined depth into the surface tissue 124 as a pulsed electric field application to induce a tissue response of the target tissue of the hollow or tubular organ. The number of electrodes 120 can be 4, 5, 6, 7, 8, 9, 10, 11, 12, 13, 14, 15, 16, 17, 18, 19, 20, 21, 22, 23, 24, 25, 26, 27, 28, 29, 30, etc. depending on the size/volume of the hollow or tubular organ 106. In some embodiments, the electrodes 120 may have between 4 and 100 electrode members, each configured to flexibly extend to contact to come proximal to the lumen wall, e.g., without losing contact. In some embodiments, the electrode members 120 are configured to extend about 1 mm-75 mm from the tubular member to have at least one electrode member (e.g., 120a, . . . 120n) positionable 0 to 5 mm from the lumen wall. The electrodes 120 may be made of a material typically used for ablation or surgical electrodes, e.g., stainless steel, titanium, nitinol. The electrodes 120 when expanded can form a basket configured to conform to the shape of the hollow or tubular organ (e.g., see FIGS. 2B, 3A, 3D, 3E, 3F).

The desired depth is typically about the first 100 to 200 microns of the cells or tissues. For the bladder, which may have a wall thickness under a centimeter, the depth may be a few microns to a millimeter. For the stomach, the depth may be 100 to several 100s microns, e.g., to get to the gastric mucosa. Deeper tissue (e.g., endothelial) in the 2-3 mm would be desired to be avoided.

Generator. The generator 110 is an electrical instrument configured, via a control program for a pulsed electric field application (e.g., electroporation, irreversible electroporation, pulsed field ablation, or targeted cell stimulation), to generate a monophasic electro pulse having a square wave, to deliver the electrical pulses to the electrodes 120. In FIG. 1A, the generator 110 is configured with a controller 126, a waveform generator 128, and an amplifier 130. The electrical instrument is configured, via a control program of the controller, to generate a set of monophasic or biphasic electropulses, e.g., per Table 2, for the irreversible electroporation and subsequent exfoliation of the surface tissue, electroporation, pulsed field ablation, and cell stimulation.

TABLE 2
Example Generator Parameters

| | |
|---|---|
| Monophasic | Voltage range of 50 V-1500 V (e.g., 50 Vₚₑₐₖ-1500 |
| | Vₚₑₐₖ) and pulse width of 10 μs-1000 μs (e.g., |
| | 50-150 μs) |
| | Interpulse delay (e.g., triggerable via an external |
| | biosignal, e.g., muscle contraction, nerve excitation, |
| | or cardiac rhythm) |
| | Pre-defined number of electropulses (e.g., having a |
| | minimum of 3-8 electropulses and a maximum of 10,000 |
| | electropulses) |
| Biphasic | Voltage range of 100 V-1500 V (e.g., 100 Vₚₚ-1500 Vₚₚ), |
| | and pulse width of 5 ns-50 μs, |
| | Interpulse delay (e.g., triggerable via an external |
| | biosignal, e.g., muscle contraction, nerve excitation, |
| | or cardiac rhythm) |
| | Pre-defined number of electropulses (e.g., having a |
| | minimum of 3-8 electropulses and a maximum of |
| | 10,000 electropulses) |

The electrical instrument (e.g., 110) may be configured, via a control program, to generate electropulses having a selectable interpulse delay (e.g., triggerable via an external biosignal, e.g., muscle contraction, nerve excitation, or cardiac rhythm). The control program may have pre-defined output configurations for irreversible electroporation and subsequent exfoliation of the surface tissue, electroporation, pulsed field ablation, cell stimulation, etc.

The electrical instrument (e.g., 110) may be configured, via a control program for irreversible electroporation, to generate biphasic electropulses comprising symmetric or asymmetric square waves.

The electrical instrument (e.g., 110) may be configured, via the control program, to generate electropulses comprising a 5 ns-1 s interpulse delay.

The electrical instrument (e.g., 110) may be configured, via the control program, to deliver a pre-defined number of electropulses (e.g., having a minimum of 3-8 electropulses and a maximum of 10,000 electropulses).

The exemplary pulsed electric field system and method can be employed for the bladder, stomach, small intestine, large intestine, blood vessels, fallopian tubes, urethra, heart, and other hollow or tubular organs, as well as ducts typically filled with fluid. FIGS. 1B and 1C show the expandable electrode catheter 102 (shown as 102b, 102c) in a tubular organ 106 (shown as 106″). FIG. 1B shows the electrodes 120 (shown as 120′) having a curved profile. FIG. 1C shows the electrodes 120 (shown as 120″) having an elongated or rectangular profile.

Example Deployment Mechanism. FIG. 1E shows an example implementation of the expandable electrode catheter 102a (shown as 102a′). In FIG. 1E, the expandable electrode catheter 102a′ includes (i) a tubular sheath 140 forming in part the tubular member 118 and (ii) an elongated shaft 142. The tubular sheath 140 has a first end 144a and a second end 144b. The shaft 142 is housed, in part, within the tubular sheath 140 and configured to be displaced (e.g., slidable) within the sheath 140. The shaft 142 has a first end 146a and a second end 146b. The second end 146b of the shaft 142 is configured to controllably move (148) to the second end 144b of the sheath to induce deployment of the electrodes 120. The electrodes 120 may be anchored (147a, 147b) to the sheath 140 and the shaft 142 so that movement between the sheath 140 and the shaft 142 causes the two anchor positions 147a, 147b to come together to induce a movement of the electrodes 120 out of the device 102a′. In FIG. 1E, the movement of the shaft 142 toward (148) the sheath 140 causes the electrodes 120 to extrude out of the device 102a′ to a deployed position, while return movement (150) of the shaft 142 from the sheath 140 causes the electrodes 120 to withdraw into the device 102a′ to a stowed position, see FIG. 3A, subpanel B.

In another embodiment (see FIG. 3A, subpanel A), the expandable electrode catheter (e.g., 102) comprises a tubular sheath (e.g., 140) forming in part the tubular member, the tubular sheath (e.g., 140) having a first end (e.g., 144a) and a second end (e.g., 144b); and a shaft (e.g., 142) housed within the tubular sheath, a portion of the tubular sheath (e.g., 140) being configured to be displaced (e.g., slidable) with respect to the shaft (e.g., 142), the shaft having a first end (e.g., 144a) and a second end (e.g., 146b), wherein the second end (e.g., 146b) of the shaft is configured to move away from the second end (e.g., 144b) of the sheath to induce deployment of the plurality of electrodes.

In FIG. 1E, the expandable electrode catheter (e.g., 102) is shown with a handle, the handle being manipulatable to move the electrodes (e.g., 120) within the hollow or tubular organ 106 (see, e.g., FIG. 3B).

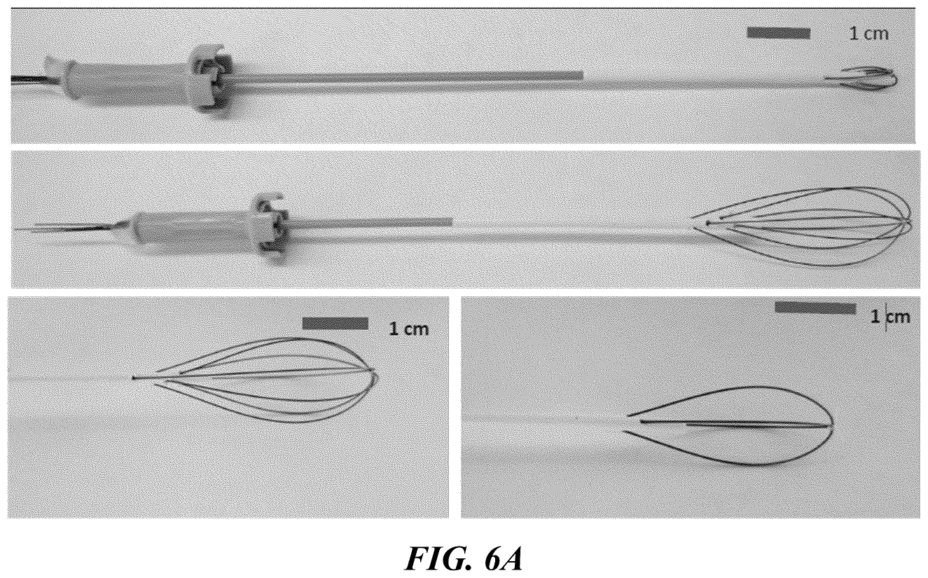

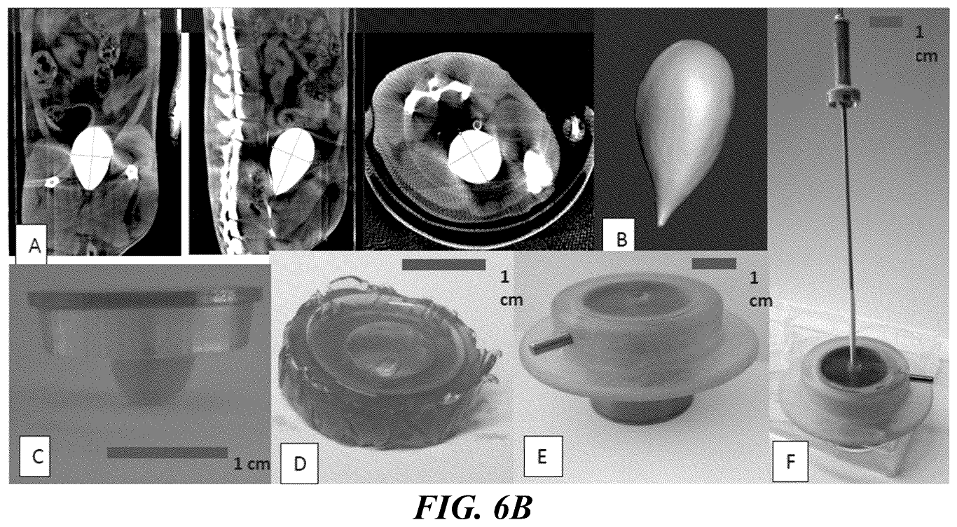

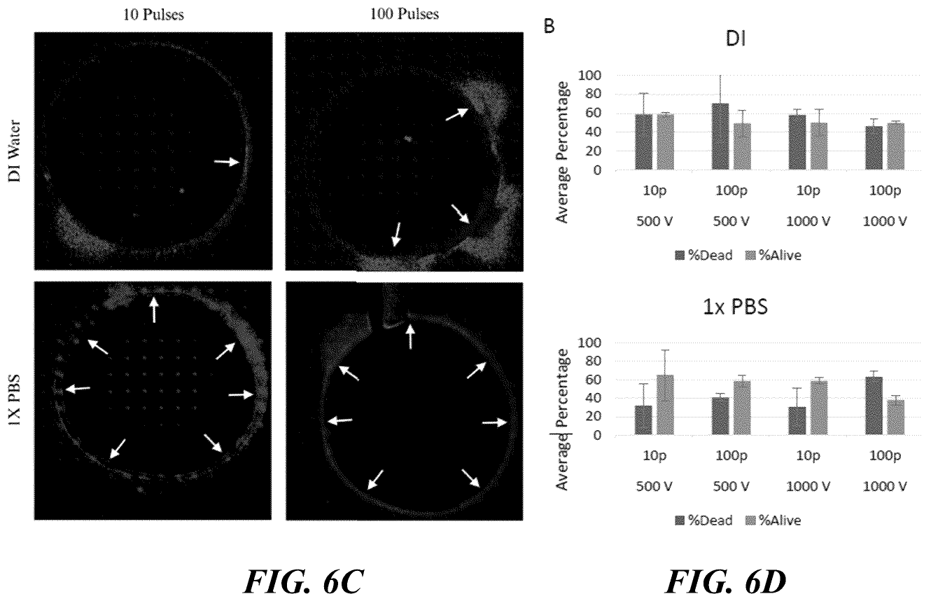

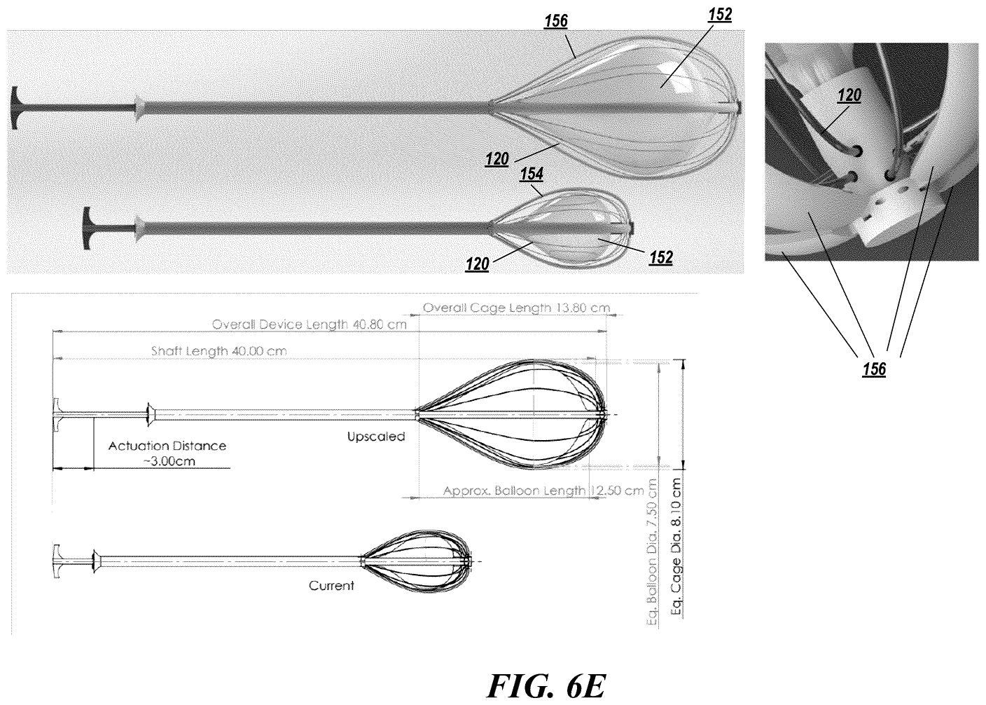

Expandable Balloon. To improve the operation of the expandable electrode catheter 102 and the controlled-conductivity liquid medium 104, the expandable electrode catheter 102 may be configured with a non-conductive expandable balloon 152 (see FIGS. 3D, 6E) that can move between a stowed position (e.g., in the tubular member 118) and a deployed position (e.g., within the envelope of the electrodes 120). With a large controlled-conductivity liquid medium 104, the liquid 104 can act as a sink/parasitic to the emitted power, requiring more energy to be provided by the generator (e.g., 110). The expandable balloon 152 is configured to expand in the volume of the hollow or tubular organ 106 to reduce the volume to be filled by the controlled-conductivity liquid medium 104. In doing so, the conductivity ratio may be better controlled as less volume is needed to be regulated. In deploying within the envelope of the electrodes 120, the electrodes 120 are positioned between the tissue 124 and the non-conductive expandable balloon 152 to direct the electrical energy towards the tissue 124.

In some embodiments, the expandable balloon 152 is an expandable sheath formed of non-conductive material, the expandable sheath is housed in one end (e.g., 122a) of the tubular member 118 and configured (i) to be stowed within the tubular member 118 in a stowed configuration and (ii) to extend out of the tubular member to define a sheath volume defined within the electrodes 120, where the sheath volume when deployed reduces a volume in a space in the hollow or tubular organ 106 for delivery of electrical pulse 108 in the liquid medium 104 to the hollow or tubular organ 106.

Expandable Non-Conductive Envelope. In some embodiments, the expandable electrode catheter (e.g., 102) further comprises an expandable non-conductive envelop 154 (also referred to as plastic basket 154) (see, e.g., FIGS. 3F, 6E) as a guide to the electrodes 120. The expandable non-conductive envelop 154 has a plurality of expandable members 156 housed at an end 122a of the tubular member 118 and configured (i) to be stowed to define an outer surface of the tubular member in a stowed configuration and (ii) to extend or expand from the tubular member to define an external envelope volume. The electrodes 120 can expand in the deployed configuration to be defined within the external envelope volume. The expandable non-conductive envelope defines a pre-defined gap or distance between the plurality of electrodes and the surface tissue.

Integrated Expandable Electrode Catheter with Conductivity Adjustment Liquid Introducer. FIG. 1D shows an example pulsed electric field system 100d configured with an expandable electrode catheter 102 (shown as 102d) configured also as a conductivity adjustment liquid introducer. In the example shown in FIG. 1D, the expandable electrode catheter 102d (shown as 102d′) includes a channel member 136 (shown as 136a, 136b) that extends, at least, between the first end and the second end of the tubular member 118 for introduction of the liquid 105, as a portion of the liquid medium 104, to be dispensed during the pulsed electric field application. The channel members 136a, 136b terminate at ports 138a, 138b. The expandable electrode catheter (e.g., 102d, 102d′) is coupled to a pump or recirculation pump 132 (shown as “Recirculation pump” 132). The recirculation loop instrument may be coupled to the expandable electrode catheter (e.g., 102) or a delivery device (e.g., 204—see FIG. 2A) to actively introduce or recirculate the liquid (105) and a portion of the liquid medium (104) during the pulsed electric field application.

FIG. 1E shows an example implementation of the expandable electrode catheter 102d (shown as 102d″). The expandable electrode catheter 102d″ includes a channel member (136a, 136b) that extends, at least, between the first end 122a and the second end 122b of the tubular member 118 for introduction of a liquid 105, as a portion of the liquid medium 104, to be dispensed during the pulsed electric field application by the expandable electrode catheter 102. The liquid 105 and liquid medium 104 can limit the focal or localized ablation of the target tissue (e.g., surface tissue 124) of the hollow or tubular organ 106 or the off-target destruction of other biological material in the hollow or tubular organ 106, such as blood or other bodily fluids. The channel members 136a, 136b has an input at ports 137a, 137b and terminates at ports 138a, 138b

In some embodiments, the device 102 is configured to operate with a liquid introducer device (not shown), the liquid introducer device being configured, via control, to introduce the liquid into the hollow or tubular organ during the pulsed electric field application and maintain the conductivity ratio in a pre-defined range, e.g., per Table 1.

### Example Instrument Configuration

FIGS. 2A-2C show example instrumentation implementation and electrode configurations for the pulsed electric field system of FIG. 1A-1E in accordance with an illustrative embodiment. FIG. 2A shows an example instrumentation implementation 200. In FIG. 2A, the expandable electrode catheter 102 is shown coupled to the generator 110 and a bioelectrical measurement instrument 202. The bioelectrical measurement instrument 202 may be used to measure the impedance of the electrodes in the liquid medium 104 to regulate or change the conductivity ratio to the range described in relation to Table 1. The generator 110 is additional shown connected to a ground pad 204 that serves as a common return/ground for the emitted electrical energy from the electrodes 120.

The instrumentation may include imaging of the expandable electrode catheter (e.g., 102) for guided delivery of the electrical pulses in the liquid medium (e.g., 104). The imaging may be used to determine to volume of the organ (e.g., 106). The imaging may employ ultrasound, X-ray, CT, MRI, and/or other imaging, endoscopic, and fluoroscopic modalities.

FIG. 2B shows example electrode configurations 208 (shown as 208a, 208b, 208c), e.g., monopolar 208a in the bladder with a distal grounding pad 206. The bipolar configuration 208b shows an example intercalated+/− electrodes (shown as 210a, 210b) and no distal grounding pad. The partial mode configuration 208c shows a small subset of electrodes 120 being electrified with reference to the grounding pad 206.

FIG. 2C shows an example electrical property measurement via the use of adjacent electrodes to measure the electrical properties of the bladder wall, before and after the pulsed electric field application.

### Mechanical Deployment of Electrodes.

FIGS. 3A-3F show different mechanical configurations for the pulsed electric field device of FIGS. 1A-1E. FIG. 3A shows two example mechanical deployment of the electrodes 120. In FIG. 3A, subpanel (a) shows a pull-back action by the external sheath being used to deploy the electrode expansion to make contact with the bladder wall and make a conforming shape. In FIG. 3A, subpanel (B) shows a push action by the external sheath being used to deploy. The external sheath is pushed forward to deploy electrodes.

FIG. 3B shows the expandable electrode catheter 102 configured with a bendable or hingeably articulatable neck 302. The catheter 102 at the entry of the bladder neck can be articulated to position the device 102 in the bladder. FIG. 3C shows the operation of the expandable electrode catheter 102 in combination with a liquid medium 104 having Ca2+, BLM, or other cell impermeable agent to increase cytotoxicity in the region of low EFS. FIG. 3D shows the expandable electrode catheter 102 configured with an internal volume balloon 152. FIG. 3E shows another example mechanical profile for the expandable electrode catheter 102, e.g., to conform to the shape of the organ (e.g., 106). FIG. 3F shows the expandable electrode catheter 102 configured with an internal volume balloon 152 and an external envelope guide 154.

### Example Method

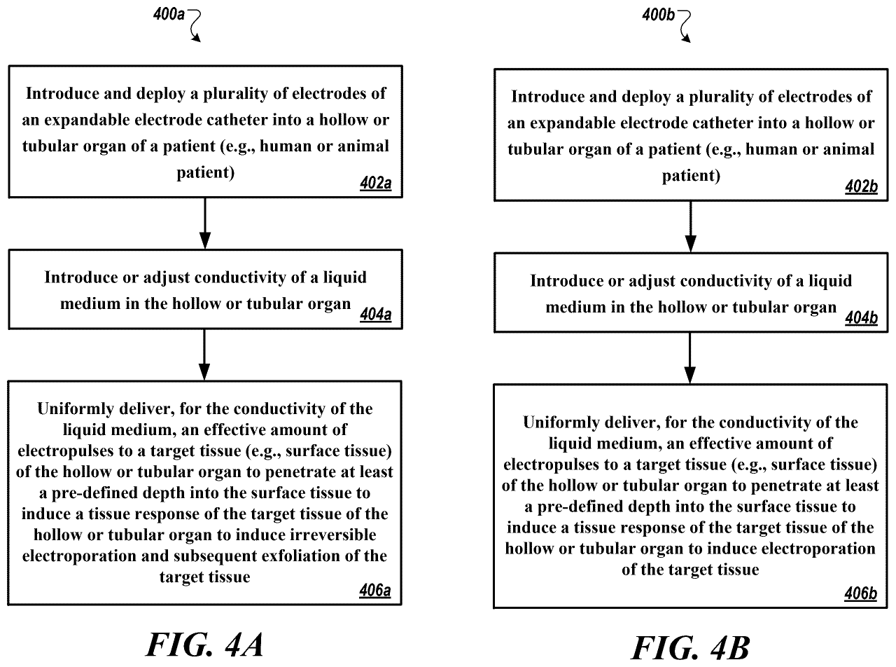

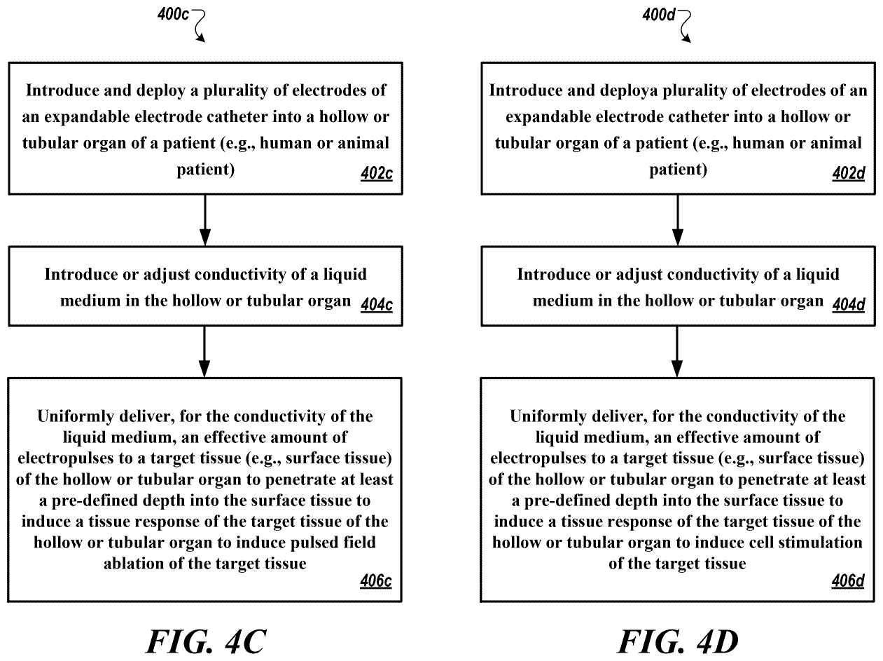

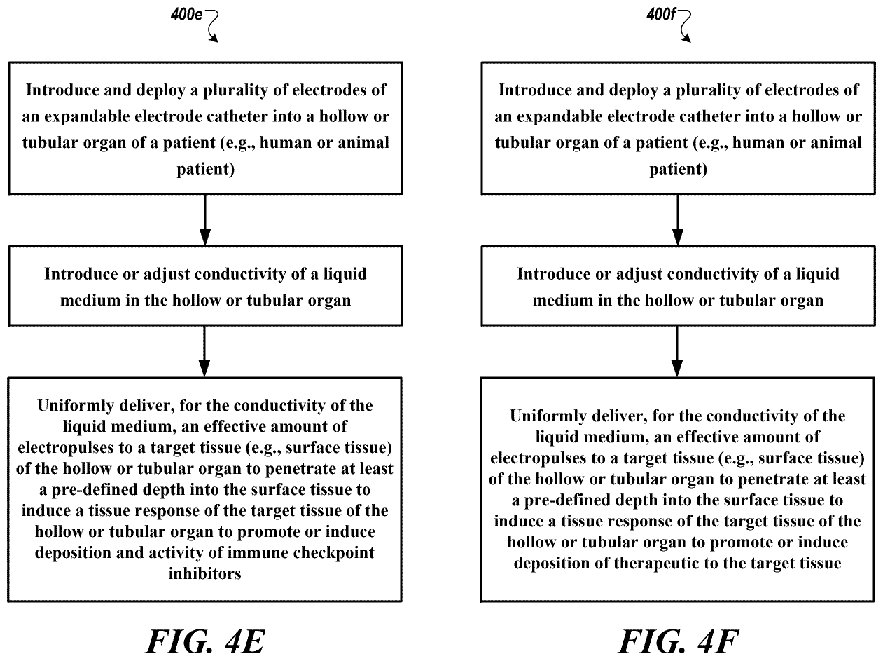

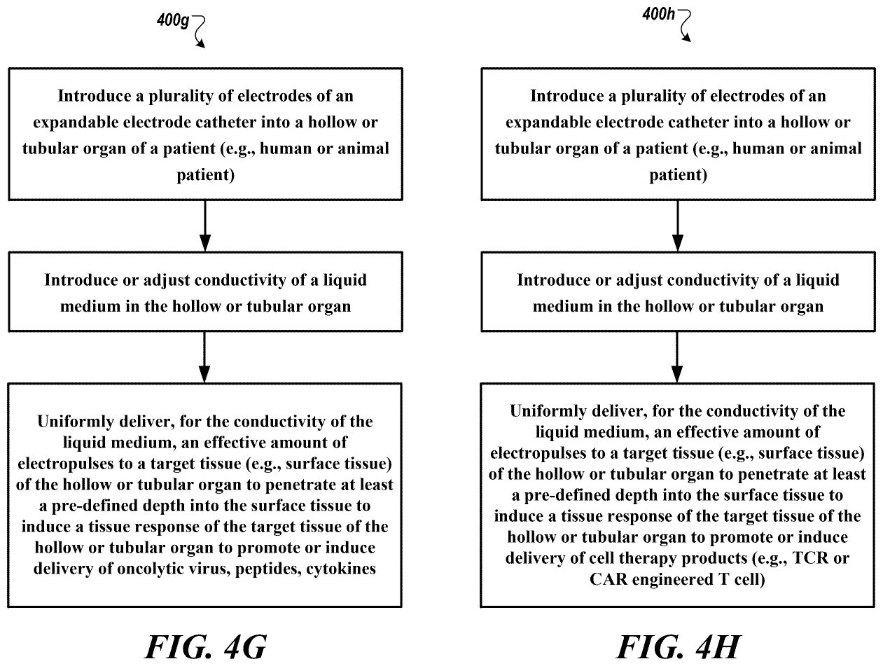

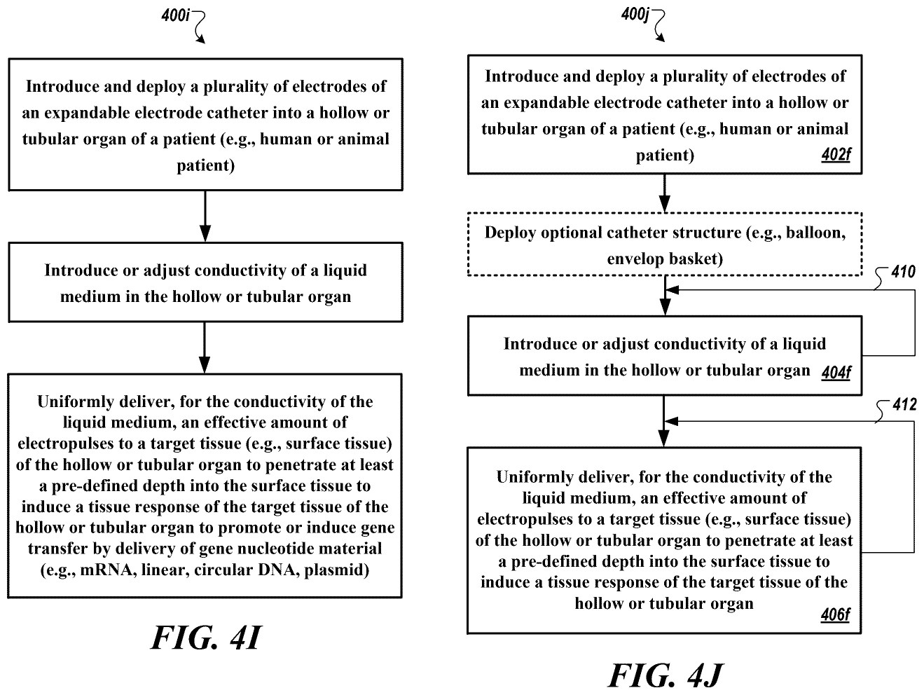

FIGS. 4A-4J show example method 400 (shown as 400a, 400b, 400c, 400d, 400e, 400f, 400g, 400h, 400i, and 400j) for the pulsed electric field application in accordance with various embodiments. The method may include a treatment with pulsed electric field application to enhance the deposition and activity of immune checkpoint inhibitors targeting PD1/PD-L1, TIM/LAG and CTLA, increase the deposition of drugs such as cisplatin, gemcitabine, mitomycin and other platinum drugs, facilitate the delivery of oncolytic virus, peptides and cytokines, facilitate the delivery of cell therapy products such as TCR or CAR engineered T cell, NK cell and macrophages, or directly allow gene transfer by delivery of mRNA, linear or circular DNA, plasmid.

Table 3 shows an example properties or control settings for the pulsed electric field to be applied in combination with (i) deposition and activity of immune checkpoint inhibitors targeting PD1/PD-L1, TIM/LAG and CTLA, (ii) deposition of drugs such as cisplatin, gemcitabine, mitomycin and other platinum drugs, (iii) delivery of oncolytic virus, peptides and cytokines, (iv) delivery of cell therapy products such as TCR or CAR engineered T cell, NK cell and macrophages, (v) gene transfer by delivery of mRNA, linear or circular DNA, plasmid.

TABLE 3

| | Example Operation Configuration |
|---|---|
| Electroporation | Apply a voltage of about 100 V-1000 V for about |
| | 10-50 pulses, monophasic or biphasic waveform, |
| | duration of electric pulses being applied about |
| | 0.001 second-0.005 second. |
| Irreversible | Apply a voltage of about 100 V-3000 V for about |
| electroporation | 5-900 pulses, monophasic or biphasic waveform, |
| | duration of electric pulses being applied about |
| | 0.01 second-0.1 second. |
| Pulsed field | Apply a voltage of about 100 V-3000 V for about |
| ablation | 5-900 pulses, monophasic or biphasic waveform, |
| | duration of electric pulses being applied about |
| | 0.01 second-0.1 second. |
| Cell | Apply voltage <500 V, biphasic waveform, duration |
| stimulation | of electric pulses being applied less than about |
| | 0.01 second. |

TABLE 4

| | Irreversible | | Pulsed Field | Cell |
|---|---|---|---|---|
| | Electroporation | Electroporation | Ablation | Stimulation |
| Cell destruction and | X | | X | |
| tissue ablation | | | | |
| Promote or induce | X | X | X | X |
| deposition and activity | | | | |
| of immune checkpoint | | | | |
| inhibitors | | | | |
| Promote or induce | X | X | X | X |
| deposition of therapeutic | | | | |
| to the target tissue | | | | |
| Promote or induce | X | X | X | X |
| delivery of oncolytic | | | | |
| virus, peptides, cytokines | | | | |
| Promote or induce | X | X | X | X |
| delivery of cell therapy | | | | |
| products (e.g., TCR or | | | | |
| CAR engineered T cell) | | | | |
| Promote or induce gene | X | X | X | X |
| transfer by delivery of | | | | |
| gene nucleotide material | | | | |
| (e.g., mRNA, linear, | | | | |
| circular DNA, plasmid) | | | | |

Table 4 shows various additional uses of pulsed electric field application using the exemplary pulsed electric field system, e.g., for electroporation, irreversible electroporation, pulsed field ablation, and cell stimulation.

Irreversible Electroporation. In FIG. 4A, method 400a includes introducing and deploying (402a) a plurality of electrodes (e.g., 120) of an expandable electrode catheter (e.g., 102) into a hollow or tubular organ (e.g., 106) of a patient (e.g., human or animal patient). The expandable extrude catheter (e.g., 102) may be any one of those described in relation to FIGS. 1A-1D, 2A-2C, and 3A-3F.

Method 400a includes introducing or adjusting (404a) the conductivity of a liquid medium (e.g., 104) in the hollow or tubular organ (e.g., 106). The adjustment (404a) may include introducing a conductive fluid (e.g., 105) (e.g., an iso-osmolar conductive fluid, or biological fluid, e.g., urine, etc.) into the hollow or tubular organ (e.g., 106) prior to or during the delivering of the electropulses. In some embodiments, method 400a includes measuring the conductivity of the liquid medium (e.g., 104) in the hollow or tubular organ (e.g., 106), e.g., via the electrodes (e.g., 120). The method 400a may include determining the conductivity ratio for the procedure, and introducing conductivity fluid until the measured conductivity is in the desired conductivity ratio, e.g., per Table 1. The operation may include removing liquid from the hollow or tubular organ, recirculating the liquid in the hollow or tubular organ while introducing fluid of greater or lower conductivity into the recirculation loop. In some embodiments, the recirculation loop may be heated or cooled to regulate the temperature of the liquid medium in the hollow or tubular organ.

Method 400a includes uniformly delivering (406a), for the conductivity of the liquid medium, an effective amount of electropulses to a target tissue (e.g., surface tissue) of the hollow or tubular organ to penetrate at least a pre-defined depth into the surface tissue to induce a tissue response of the target tissue of the hollow or tubular organ to induce irreversible electroporation and subsequent exfoliation of the target tissue as a tissue response to the pulsed electric field application, e.g., per Tables 2 or 3.

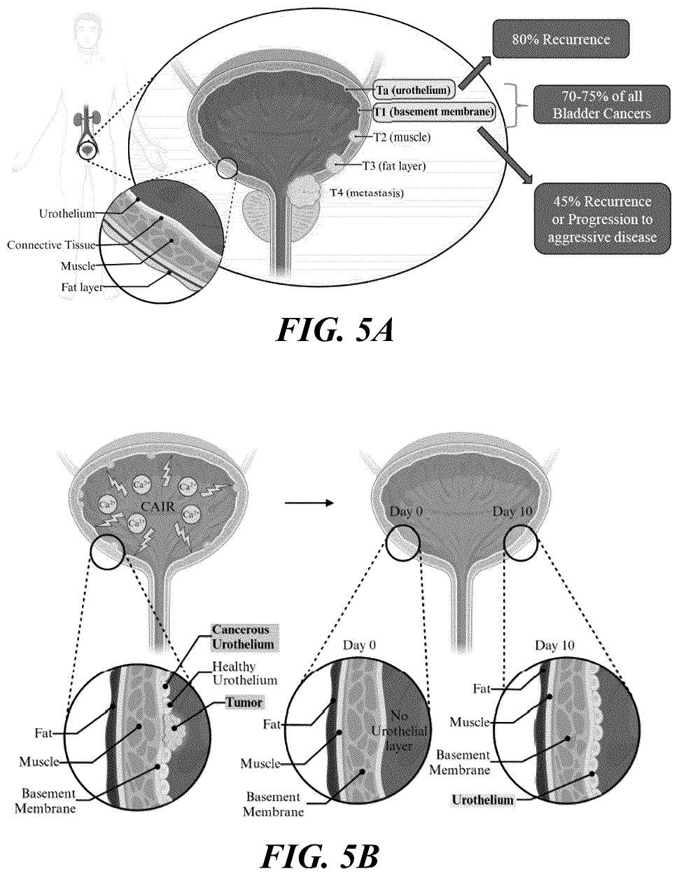

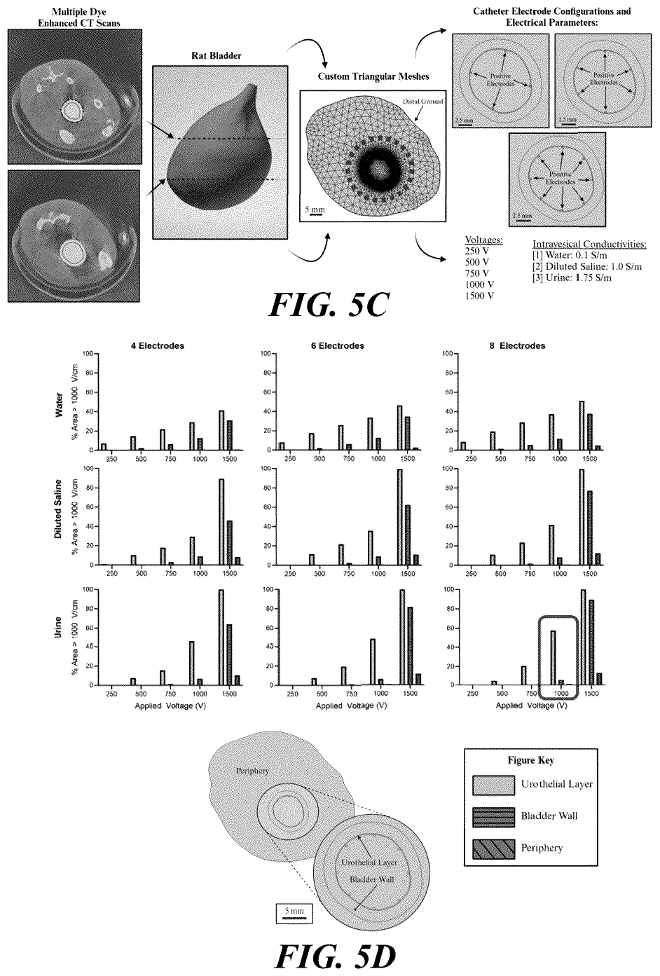

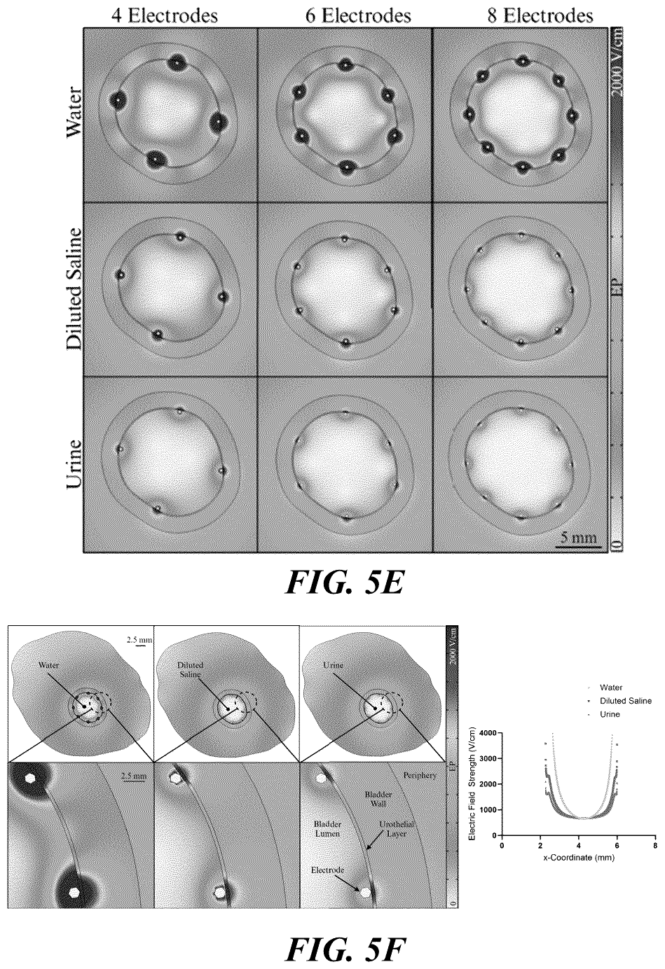

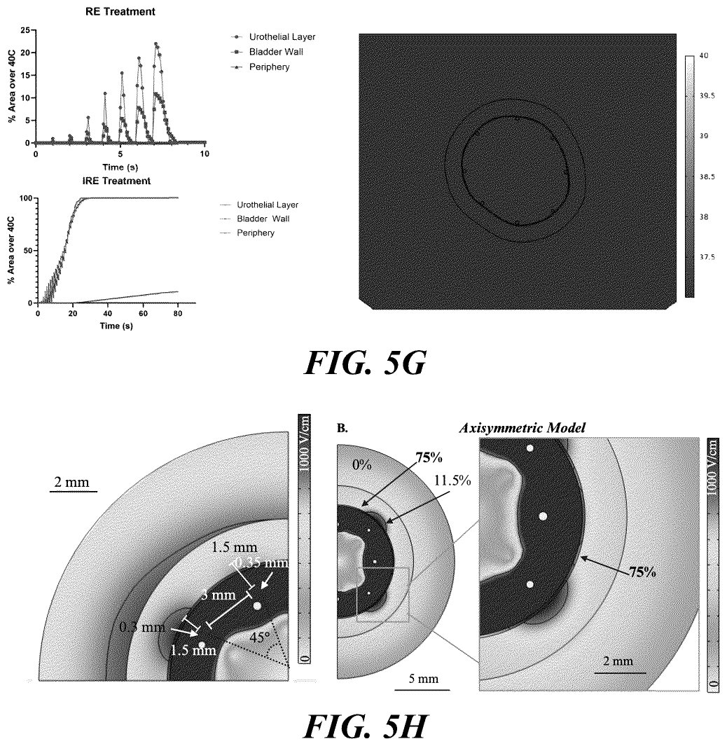

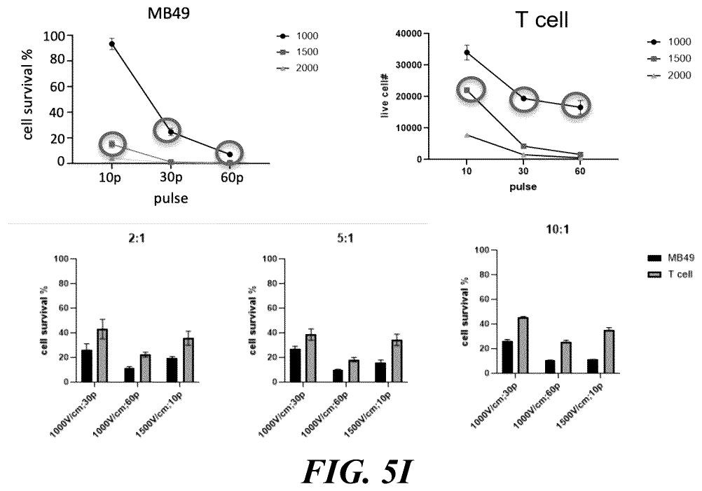

The partial or full epithelial or urothelial tissue exfoliation may be used to treat cancer. For bladder cancer, the pulse configurations may be selected to selectively induce a response in the epithelial or cancer cells and a lesser response on T cells (e.g., sparing one cell type, e.g., T cells while killing epithelial or cancer cells), e.g., as described in relation to FIG. 5I.

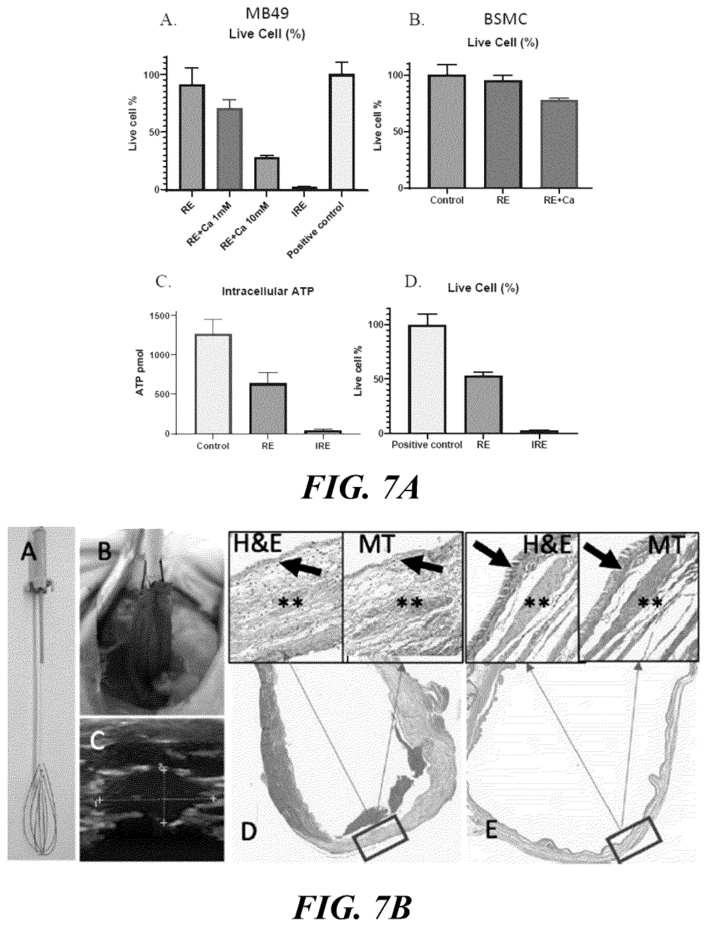

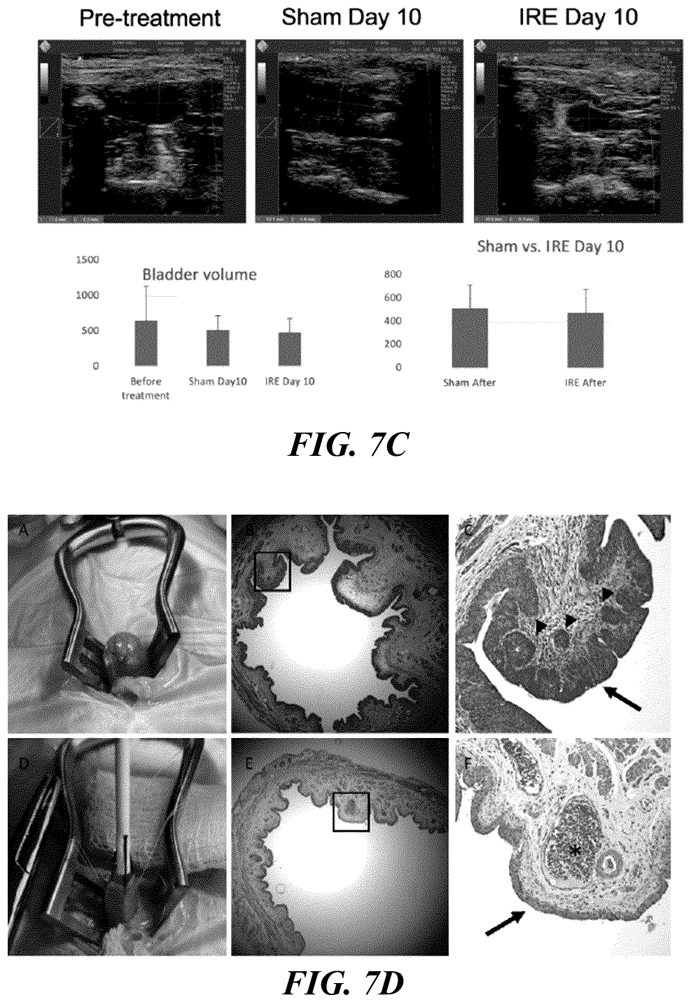

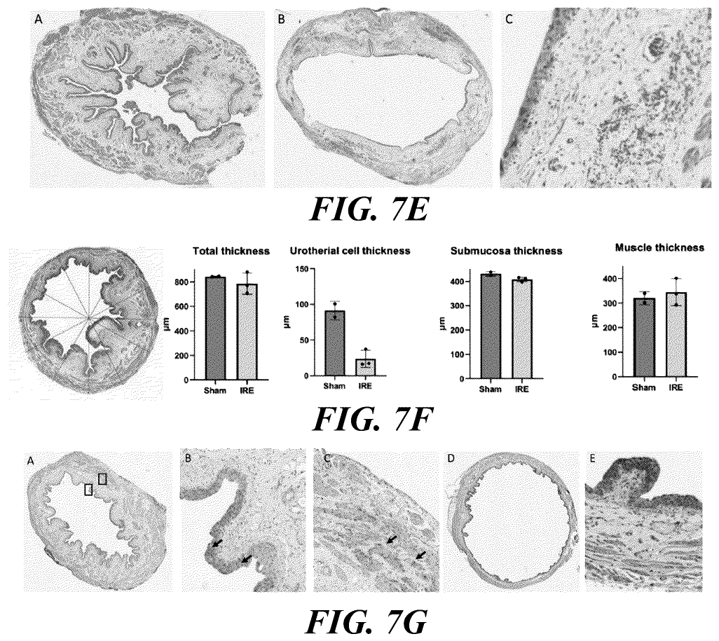

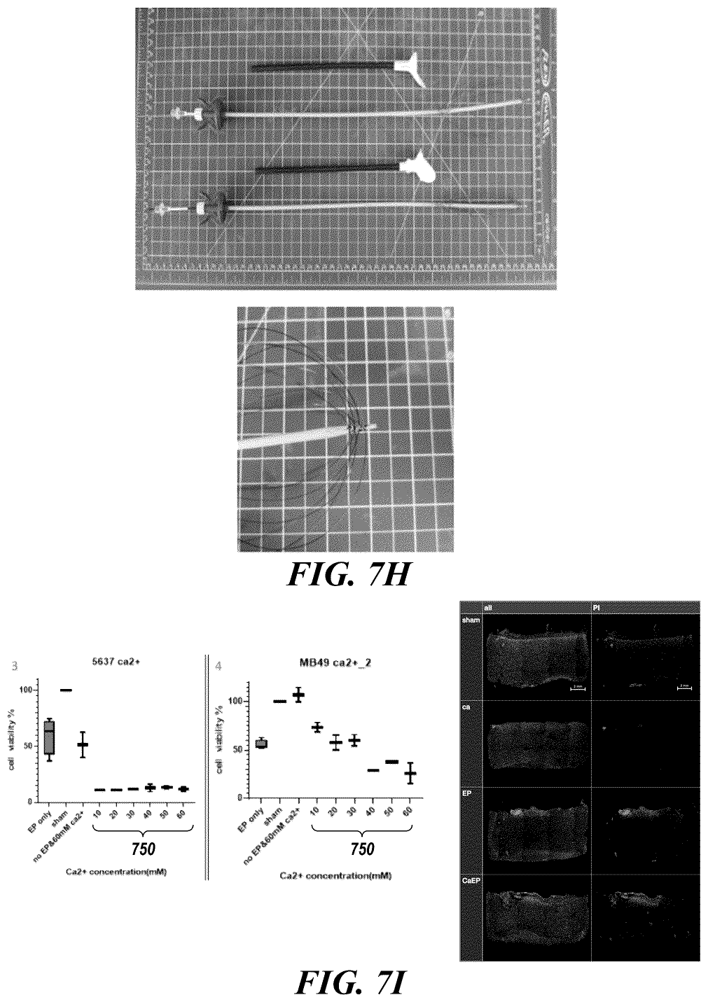

In some embodiments, Method 400a may include introducing a cell impermeable agent prior to or during the delivery of the electropulses to increase cytotoxicity in the hollow or tubular organ. The cell impermeable agent may be calcium (e.g., Ca2+), bleomycin, cisplatin, or a combination thereof, among other cell impermeable agents described or referenced herein, e.g., as described in relation to FIGS. 7A-7I.

The partial or full epithelial or urothelial tissue exfoliation of the full organ (or a large region of it) may be to (i) promote or induce deposition and activity of immune checkpoint inhibitors targeting PD1/PD-L1, TIM/LAG and CTLA at the hollow or tubular organ (FIG. 4E), (ii) promote or induce deposition of therapeutics (e.g., cisplatin, gemcitabine, mitomycin and other platinum drugs) at the hollow or tubular organ (FIG. 4F), (iii) promote or induce delivery of oncolytic virus, peptides, cytokines, or a combination thereof at the hollow or tubular organ (FIG. 4G), (iv) promote or induce delivery of cell therapy products such as TCR or CAR engineered T cell, NK cell and macrophages at the hollow or tubular organ (FIG. 4H), or (v) promote or induce gene transfer by delivery of mRNA, linear, circular DNA, plasmid, or a combination thereof at the hollow or tubular organ (FIG. 4I).

Electroporation. In FIG. 4B, method 400b includes introducing and deploying (402b) a plurality of electrodes (e.g., 120) of an expandable electrode catheter (e.g., 102) into a hollow or tubular organ (e.g., 106) of a patient (e.g., human or animal patient). The expandable electrode catheter (e.g., 102) may be any one of those described in relation to FIGS. 1A-1D, 2A 2C, and 3A-3F.

Method 400b includes introducing or adjusting (404b) the conductivity of a liquid medium (e.g., 104) in the hollow or tubular organ (e.g., 106). The adjustment (404b) may include introducing a conductive fluid (e.g., 105) (e.g., an iso-osmolar conductive fluid, or biological fluid, e.g., urine, etc.) into the hollow or tubular organ (e.g., 106) prior to or during the delivering of the electropulses. In some embodiments, method 400b includes measuring the conductivity of the liquid medium (e.g., 104) in the hollow or tubular organ (e.g., 106), e.g., via the electrodes (e.g., 102). The method 400b may include determining the conductivity ratio for the procedure, and introducing conductivity fluid until the measured conductivity is in the desired conductivity ratio, e.g., per Table 1. The operation may include removing liquid from the hollow or tubular organ, recirculating the liquid in the hollow or tubular organ while introducing fluid of greater or lower conductivity into the recirculation loop. In some embodiments, the recirculation loop may be heated or cooled to regulate the temperature of the liquid medium in the hollow or tubular organ.

Method 400b includes uniformly delivering (406b), for the conductivity of the liquid medium, an effective amount of electropulses to a target tissue (e.g., surface tissue) of the hollow or tubular organ to penetrate at least a pre-defined depth into the surface tissue to induce a tissue response of the target tissue of the hollow or tubular organ to induce electroporation of the target tissue, e.g., per Tables 2 or 3.

The partial or full electroporation of the whole organ or a large region of the organ may be to (i) promote or induce deposition and activity of immune checkpoint inhibitors targeting PD1/PD-L1, TIM/LAG and CTLA at the hollow or tubular organ (FIG. 4E), (ii) promote or induce deposition of therapeutics (e.g., cisplatin, gemcitabine, mitomycin and other platinum drugs) at the hollow or tubular organ (FIG. 4F), (iii) promote or induce the delivery of oncolytic virus, peptides, cytokines, or a combination thereof at the hollow or tubular organ (FIG. 4G), (iv) promote or induce the delivery of cell therapy products such as TCR or CAR engineered T cell, NK cell and macrophages at the hollow or tubular organ (FIG. 4H), or (v) promote or induce gene transfer by delivery of mRNA, linear, circular DNA, plasmid, or a combination thereof at the hollow or tubular organ (FIG. 4I).

Pulsed Field Ablation. In FIG. 4C, method 400c includes introducing and deploying (402c) a plurality of electrodes (e.g., 120) of an expandable electrode catheter (e.g., 102) into a hollow or tubular organ (e.g., 106) of a patient (e.g., human or animal patient). The expandable extrude catheter (e.g., 102) may be any one of those described in relation to FIGS. 1A-1D, 2A-2C, and 3A-3F.

Method 400c includes introducing or adjusting (404c) the conductivity of a liquid medium (e.g., 104) in the hollow or tubular organ (e.g., 106). The adjustment (404c) may include introducing a conductive fluid (e.g., 105) (e.g., an iso-osmolar conductive fluid, or biological fluid, e.g., urine, etc.) into the hollow or tubular organ (e.g., 106) prior to or during the delivering of the electropulses. In some embodiments, method 400c includes measuring the conductivity of the liquid medium (e.g., 104) in the hollow or tubular organ (e.g., 106), e.g., via the electrodes (e.g., 120). The method 400c may include determining the conductivity ratio for the procedure, and introducing conductivity fluid until the measured conductivity is in the desired conductivity ratio, e.g., per Table 1. The operation may include removing liquid from the hollow or tubular organ, recirculating the liquid in the hollow or tubular organ while introducing fluid of greater or lower conductivity into the recirculation loop. In some embodiments, the recirculation loop may be heated or cooled to regulate the temperature of the liquid medium in the hollow or tubular organ.

Method 400c includes uniformly delivering (406c), for the conductivity of the liquid medium, an effective amount of electropulses to a target tissue (e.g., surface tissue) of the hollow or tubular organ to penetrate at least a pre-defined depth into the surface tissue to induce a tissue response of the target tissue of the hollow or tubular organ to induce pulsed field ablation of the target tissue as a tissue response to the pulsed electric field application, e.g., per Tables 2 or 3.

In some embodiments, Method 400c may include introducing a cell impermeable agent prior to or during the delivery of the electropulses to increase cytotoxicity in the hollow or tubular organ. The cell impermeable agent may be calcium (e.g., Ca2+), bleomycin, cisplatin, or a combination thereof, among other cell impermeable agents described or referenced herein, e.g., as described in relation to FIGS. 7A-7I.

The partial or full epithelial pulsed field ablation, e.g., of cardiac tissue may be to (i) promote or induce deposition and activity of immune checkpoint inhibitors targeting PD1/PD-L1, TIM/LAG and CTLA at the hollow or tubular organ (FIG. 4E), (ii) promote or induce deposition of therapeutics (e.g., cisplatin, gemcitabine, mitomycin and other platinum drugs) at the hollow or tubular organ (FIG. 4F), (iii) promote or induce delivery of oncolytic virus, peptides, cytokines, or a combination thereof at the hollow or tubular organ (FIG. 4G), (iv) promote or induce delivery of cell therapy products such as TCR or CAR engineered T cell, NK cell and macrophages at the hollow or tubular organ (FIG. 4H), or (v) promote or induce gene transfer by delivery of mRNA, linear, circular DNA, plasmid, or a combination thereof at the hollow or tubular organ (FIG. 4I).

The exemplary pulsed electric field system can address hemolysis by targeting the vascular lumen walls without causing damage to the blood and blood cells.

Cell Stimulation. In FIG. 4D, method 400d includes introducing and deploying (402d) a plurality of electrodes (e.g., 120) of an expandable electrode catheter (e.g., 102) into a hollow or tubular organ (e.g., 106) of a patient (e.g., human or animal patient). The expandable electrode catheter (e.g., 102) may be any one of those described in relation to FIGS. 1A-1D, 2A-2C, and 3A-3F.

Method 400d includes introducing or adjusting (404d) the conductivity of a liquid medium (e.g., 104) in the hollow or tubular organ (e.g., 106). The adjustment (404d) may include introducing a conductive fluid (e.g., 105) (e.g., an iso-osmolar conductive fluid, or biological fluid, e.g., urine, etc.) into the hollow or tubular organ (e.g., 106) prior to or during the delivering of the electropulses. In some embodiments, method 400d includes measuring the conductivity of the liquid medium (e.g., 104) in the hollow or tubular organ (e.g., 106), e.g., via the electrodes (e.g., 102). The method 400d may include determining the conductivity ratio for the procedure, and introducing conductivity fluid until the measured conductivity is in the desired conductivity ratio, e.g., per Table 1. The operation may include removing liquid from the hollow or tubular organ, recirculating the liquid in the hollow or tubular organ while introducing fluid of greater or lower conductivity into the recirculation loop. In some embodiments, the recirculation loop may be heated or cooled to regulate the temperature of the liquid medium in the hollow or tubular organ.

Method 400d includes uniformly delivering (406d), for the conductivity of the liquid medium, an effective amount of electropulses to a target tissue (e.g., surface tissue) of the hollow or tubular organ to penetrate at least a pre-defined depth into the surface tissue to induce a tissue response of the target tissue of the hollow or tubular organ to induce electroporation of the target tissue, e.g., per Tables 2 or 3.

The partial or full cell stimulation of the whole organ or a large region of the organ may be to (i) promote or induce deposition and activity of immune checkpoint inhibitors targeting PD1/PD-L1, TIM/LAG and CTLA at the hollow or tubular organ (FIG. 4E), (ii) promote or induce deposition of therapeutics (e.g., cisplatin, gemcitabine, mitomycin and other platinum drugs) at the hollow or tubular organ (FIG. 4F), (iii) promote or induce delivery of oncolytic virus, peptides, cytokines, or a combination thereof at the hollow or tubular organ (FIG. 4G), (iv) promote or induce delivery of cell therapy products such as TCR or CAR engineered T cell, NK cell and macrophages at the hollow or tubular organ (FIG. 4H), or (v) promote or induce gene transfer by delivery of mRNA, linear, circular DNA, plasmid, or a combination thereof at the hollow or tubular organ (FIG. 4I).

FIG. 4E shows the exemplary system and method may be used to promote or induce deposition and activity of immune checkpoint inhibitors targeting PD1/PD-L1, TIM/LAG, and CTLA at the hollow or tubular organ.

FIG. 4F shows the exemplary system and method may be used to promote or induce the deposition of therapeutics (e.g., cisplatin, gemcitabine, mitomycin, and other drugs) at the hollow or tubular organ.

FIG. 4G shows the exemplary system and method may be used to promote or induce the delivery of oncolytic virus, peptides, cytokines, or a combination thereof at the hollow or tubular organ.

FIG. 4H shows the exemplary system and method may be used to promote or induce the delivery of cell therapy products, such as TCR or CAR-engineered T cells, NK cells, and macrophages at the hollow or tubular organs.

FIG. 4I shows the exemplary system and method may be used to promote or induce gene transfer by the delivery of mRNA, linear, circular DNA, plasmid, or a combination thereof at the hollow or tubular organ.

FIG. 4F shows an example method 400f for controlling the operation of the pulsed electric device of any one of FIGS. 1A-1E. Method 400f includes introducing and deploying (402f) a plurality of electrodes (e.g., 120) of an expandable electrode catheter (e.g., 102) into a hollow or tubular organ (e.g., 106) of a patient (e.g., human or animal patient). The expandable extrude catheter (e.g., 102) may be any one of those described in relation to FIGS. 1A-1D, 2A-2C, and 3A-3F.

Method 400f shows an optional operation to employ, e.g., the balloon 152, or guided cage 154 as described in relation to FIGS. 3D, 3F, among others.

Method 400f includes introducing or adjusting (404c) the conductivity of a liquid medium (e.g., 104) in the hollow or tubular organ (e.g., 106). The adjustment (404c) may include introducing a conductive fluid (e.g., 105) (e.g., an iso-osmolar conductive fluid, or biological fluid, e.g., urine, etc.) into the hollow or tubular organ (e.g., 106) prior to or during the delivering of the electropulses. In some embodiments, method 400f includes measuring the conductivity of the liquid medium (e.g., 104) in the hollow or tubular organ (e.g., 106), e.g., via the electrodes (e.g., 120). The method 400f may include determining the conductivity ratio for the procedure, and introducing conductivity fluid until the measured conductivity is in the desired conductivity ratio, e.g., per Table 1. The operation may include removing liquid from the hollow or tubular organ, recirculating the liquid in the hollow or tubular organ while introducing fluid of greater or lower conductivity into the recirculation loop. In some embodiments, the recirculation loop may be heated or cooled to regulate the temperature of the liquid medium in the hollow or tubular organ. The adjustment 404c may be performed in an ongoing manner (410), e.g., via a closed-loop control operation. In some embodiments, the adjustment is performed once, e.g., at the beginning of the procedure.

Method 400f includes uniformly delivering (406c), for the conductivity of the liquid medium, an effective amount of electropulses to a target tissue (e.g., surface tissue) of the hollow or tubular organ to penetrate at least a pre-defined depth into the surface tissue to induce a tissue response of the target tissue of the hollow or tubular organ to induce pulsed field ablation of the target tissue as a tissue response to the pulsed electric field application, e.g., per Tables 2 or 3. The adjustment to the delivery may be performed in an on-going manner (412), e.g., via a closed-loop control operation.

In some embodiments, Method 400f may include introducing a cell impermeable agent prior to or during the delivery of the electropulses to increase cytotoxicity in the hollow or tubular organ. The cell impermeable agent may be calcium (e.g., Ca2+), bleomycin, cisplatin, or a combination thereof, among other cell impermeable agents described or referenced herein, e.g., as described in relation to FIGS. 7A-7I.

### Experimental Results and Additional Examples

A study was conducted to develop and evaluate a basket electrode catheter to deliver pulsed electric field (PEF) treatment to the entire bladder. This device includes a collapsable electrode basket and balloon catheter that can be inserted into the bladder through the urethra and deployed to ablate the bladder wall. The study developed computational models to optimize electrode design and predict treatment penetration, and in-vitro and in-vivo experiments validate computational findings. The key design characteristics and associated results are described below.

Whole Bladder Pulsed Electric Field (PEF) Treatment. FIG. 5A shows an example of stages of bladder cancer. FIG. 5B shows an illustration of the system and method employed in the study to use Pulsed Electric Field (PEF) to treat the bladder, completely or partially resurfacing the entire top layer of cells, cancerous and healthy, while maintaining cell viability in the remaining layers of the bladder wall.

Bladder is the fourth most common site of cancer in men with an estimated annual incidence of 81,400 new cases for both sexes [1], [2]. Bladder cancer (Bladder cancer) is diagnosed at an early and localized stage in over 75% of the patients [3], [4], presenting as non-muscle invasive tumors (NMIBC) or carcinoma in situ (CIS). Preference is given to minimally invasive treatments that preserves normal urinary function in these patients [5]. However, the majority of Bladder cancer patients will experience local tumor recurrence despite standard of care transurethral resection of bladder tumor (TURBT) and adjuvant intravesical immuno- or chemotherapy (FIG. 5A).

Recurrence is observed in 55-61% of patients within one year after commencing treatment [6], [7], increasing to 78% at 5 years from initial diagnosis [6]. The propensity for, and the timeline to recurrence, is unpredictable [8], necessitating annual cystoscopy examinations and multiple cycles of adjuvant intravesical therapy. While necessary to ensure patient survival, this clinical paradigm imposes a considerable burden on the healthcare system as well as the patient's mental well-being.

Patients with high-grade recurrence or treatment-resistant disease are at elevated risk of developing metastatic cancer [8], [9], and are recommended to undergo radical cystectomy (RC) [10], [11]. While RC is curative, potential benefits have to be balanced with the high risk of morbidity (60%) and 30-day surgery-related mortality (2.9%) [12], [13], [14]. Adoption of enhanced recovery after surgery (ERAS) protocols can mitigate morbidity following RC [15], [16], yet loss of urinary continence and sexual function severely impacts patients for the remainder of their survival [17], [18]. Curative treatment of localized Bladder cancer remains elusive despite early detection in the majority of patients with the disease. Availability of non-surgical interventions that can effectively prevent post-TURBT local recurrence is an unmet clinical need.

The etiology of bladder cancer results in widespread disease foci from intravesical or trans-epithelial migration of urothelial cancer cells (UCC) [19], and “field cancerization effect” from exposure to carcinogens. Clinical studies reveal the presence of multiple cancer fields in a patient's bladder at initial diagnosis, with driver mutations present in even normal appearing urothelium [20]. The clinical paradigm, therefore, presumes that even normal appearing urothelium of the patient may harbor diseased cells with tumorigenic potential.

Rigor of Prior Research and Current Standard of Care. Tumor resection and ablation during TURBT have been established to be effective for the treatment of Bladder cancer visible on endoscopic imaging. Intravesical chemotherapy or Bacille Calmette Guerin (BCG) vaccine immunotherapy is then performed to mitigate recurrence from microscopic tumors or diseased cells dispersed within the urothelial layer. The importance of adjuvant intravesical chemotherapy in reducing Bladder cancer recurrence in comparison to TURBT alone was confirmed by the results of the recently concluded SWOG S0337 trial [21]. Intravesical chemotherapy using mitomycin C [22], gemcitabine [21], [23], cisplatin [24], valrubicin [25], and paclitaxel [26] has been reported in the literature, with modest benefit in disease or progression-free survival. A high proportion of non-responsive patients²² and the emergence of treatment-resistant disease [27], [28], [24] are common challenges to intravesical chemotherapy of Bladder cancer. Hyperthermia [29] and electromotive drug application have been evaluated in patients to improve intravesical chemotherapy, with modest improvements in outcomes at best.

Intravesical BCG administration has been used to control BLADDER CANCER recurrence for many decades and is generally considered the most effective first line of therapy [5], [31], [32]. While many patients initially respond to BCG therapy, recurrence within two years is expected in 48% of patients, and a considerable portion of these patients progress to invasive cancer [33]. Outcomes of BCG therapy are especially sensitive to the strain of bacterium used, timing between cycles, and the need for induction therapy followed by maintenance at 6-12 month intervals, complicating uniform clinical practice [34]. Novel therapeutic strategies such as cytokine administration [35], immune checkpoint inhibitors [36] (ICI), and antibody-drug conjugates [37] (ADCC) have been examined in patients at high-risk of recurrence or BCG refractory disease. Even the most promising of these therapies yielded a complete response rate of 45.5% at one year and 38.8% at three months for nadofaragene firadenovec [37] and pembrolizumab [36], respectively, with progressive disease recurrence following that initial period. Low complete response rate, emergence of treatment-resistant or invasive cancer, the lack of durable cancer control and treatment-related toxicity are obvious gaps in adjuvant therapies used to control post-TURBT Bladder cancer recurrence, underscoring the need for newer approaches that can surmount these challenges. Our objective is to address this critical gap in Bladder cancer therapy by developing new technology for image-guided whole bladder resurfacing of urothelium with irreversible electroporation (IRE), creating a novel approach for abrogating tumor recurrence.

Computational Models to Optimize Basket Design and Predict Treatment Penetration. FIG. 5C-5I shows computation simulation results of modeling analysis to design a pulsed electric field device. FIG. 5C shows a computational simulation workflow to guide the design of catheter device geometry and electric pulse parameter optimization.

Contrast-enhanced computational tomography (CT) images of a rat bladder were used to construct finite element models to optimize the size, distribution, and configuration of the electrode basket, along with electric pulse parameters required for whole-bladder exfoliation of the urothelial layer. Stationary and time-dependent models were built with 4, 6, and 8 electrodes contacting the bladder, and a parameter sweep was conducted with a range of intravesical conductivities (0.1-1.75 S/m) and applied voltages (250-1500 V). The urothelial layer was explicitly modeled using a biologically accurate thickness from previously published data. The mesh of the urothelial was strictly built, ensuring sufficient elements across the domain with a maximum element size of 0.01 mm and a maximum element growth factor of 1.1 (FIG. 5C). The electrodes were set as the positive DC voltage boundary conditions and ground was assumed to be a distal ground pad on the perimeter of the domains. The electric field strength (EFS) was calculated using Maxwell's and Current Conservation Equations, and magnetism was assumed to be negligible.

Computational simulations show that increasing the quantity of electrodes can increase the EFS distribution across the urothelial layer but also increase the amount of current draw. Intravesical conductivity (0.1-1.75 S/m) is inversely proportional to penetration and intensity of EFS in the urothelial layer and the EFS gradient between electrodes (17% and 100% reduction, respectively), and directly proportional to current drawn (35%) (FIGS. 5D-5F). While electroporation is commonly considered non-thermal, our simulations show that tissue directly adjacent to the electrodes can experience temperature increase due to Joule heating (FIG. 5G).

FIG. 5D shows simulation results for several amplitudes, showing that increasing electrode numbers and increasing intravesical conductivity can improve electroporation performance. FIG. 5E shows simulation results with 1000V amplitude, showing that increasing electrode quantity and increasing intravesical conductivity can improve electroporation performance based on coverage of the urothelial lining without deeper penetration. FIG. 5F shows conductivity increase results in a more uniform electric field strength of the urothelial layer between electrodes.

FIG. 5H shows that the careful selection of bladder wall to lumen conductivity gradient can enable non-contact application of electric fields with greater coverage of urothelium without heating or high-intensity artifact that can arise from direct electrode-tissue contact.

FIG. 5I shows that the careful selection of electric pulse parameters can enable greater sparing of T cells while killing bladder cancer MB49 cells. This effect is accentuated even when the two cells are in different ratios, as commonly found in the tumor microenvironment.

PEF Delivery in Tumor Mimics to assess IRE Ablation Regions. Simulation results were validated in vitro by performing IRE with prototype basket electrode catheters (FIG. 6A). FIG. 6A shows two different embodiments of a catheter device used for in vitro testing of tumor mimic hydrogels and in rat models. To create the 3D rat bladder gel phantoms, CT scans of a rat were taken at various angles, then a 3D reconstruction of the bladder was built and 3D printed to create a positive mold and respective PDMS negative mold. Agarose embedded with murine bladder cancer (MB49) cells was then seeded in the PDMS mold and treated with EP (FIG. 6B). PI staining and microscopy imaging were used to map the IRE penetration and distribution of ablation. FIG. 6B shows a 3D Rat bladder tumor mimic workflow and EP treatment approach.

Results demonstrate that depth and penetration of ablation increase with PEF dose and intravesical conductivity (2.4:1) and are linked to heterogeneity in the depth of penetration (FIGS. 6C and 6D). FIGS. 6C and 6D show IRE depth and uniformity increase with pulse number and conductivity in vitro;

FIG. 6E shows an alternative embodiment of a second-generation catheter device incorporating a balloon to reduce the volume of conductivity fluid within the bladder and mitigate current drawn/heating during treatment. A plastic spacer is incorporated to avoid direct contact of electrodes with the bladder wall while maintaining uniform penetration and urothelial targeting. The electrode size, geometry, and configuration are informed by simulation models to cover the entire urothelial lining of the bladder wall.

Calcium Assisted Irreversible Electroporation (CAIR) to enhance EFS Uniformity. The study employed calcium to increase lethality even at sub-therapeutic energy doses and to increase the depth of penetration of the pulsed electric field application. With the inclusion of calcium, the conductivity of the medium can be adjusted so that the electrode can operate without contact to the lumen, to reduce thermal damage.

Intracellular Ca²⁺ is carefully regulated by cells, as even a transient increase in levels can trigger cell death. While transmembrane transport of Ca²⁺ is controlled by ion channels and pumps, extracellular Ca²⁺ can freely enter the cell when the membrane is in a permeabilized state during IRE, or even during conditions of short-term transient membrane permeabilization from reversible electroporation (RE). Frandsen et al.³⁵ were first to take advantage of this observation to induce cell death by performing RE in the presence of exogenous Ca²⁺, where RE or exogenous Ca²⁺ alone does not have considerable cytotoxicity³⁶. Cells exhibit considerable differences in their ability to tolerate intracellular Ca²⁺, where BSMCs can experience a 200,000-fold shift in Ca²⁺ levels during normal physiologic activity (muscle contraction, etc.) and have the capacity to rapidly transport the excess Ca²⁺ to stores in the sarcoplasmic reticulum.

In the data of the study, it can be observed that RE in the presence of Ca²⁺ can produce cell death comparable to IRE of bladder cancer cells (MB49), thereby allowing a 90% reduction in the electrical energy required for treatment (10 pulses for RE, 100 pulses for IRE). Further, the results show that Ca²⁺ provokes more toxicity in MB49 cells than BSMC, which is correlated to intracellular ATP loss (FIG. 7A). By exploiting the intercellular differences between BSMC and UCC in their ability to tolerate increased Ca²⁺ levels, we will advance a new technique to increase the specificity and efficacy of necrotic cell death to the latter cell type, while reducing the magnitude of electrical energy and time required to perform the treatment. Specifically, FIG. 7A shows, in the top panels, the treatment with Ca2+ and RE and, in the bottom panels, ATP depletion and corresponding cell survival after IRE of MB49 cells. FIG. 7A, subpanel (a) shows that MB49 cells exhibit considerable loss of viability when combining RE with Ca2+, with greater cell death proportional to the concentration of exogenous Ca2+; subpanel (b) shows BSMC treated with RE and 10 mM CaCl2 demonstrating greater cell survival than UCC. FIG. 7A, subpanel (c) shows intracellular ATP depletion having a correlation with electrical energy used, with more loss for IRE than RE; subpanel (d) shows cell survival correlating with both ATP loss and energy dose applied to cells.

The study tested the efficacy and safety of CAIR with healthy Sprague Dawley (n=20) and cancer (n=22) bearing Wistar rats (by exposure to N-butyl-N-(2-hydroxylbutyl) nitrosamine in water). The bladder was surgically accessed for placement of the electrode catheter and was filled with 5 mmol CaCl2+0.9% saline solution prior to treatment (1000 V/cm, 100 μs pulse length, 100 pulses, 1 Hz, 2× pulsing with repositioning of the electrodes). Rats were clinically monitored prior to sacrifice at 1, 7, and 10 days post-treatment. Bladders were processed for histology with Hematoxylin and Eosin, Masson's Trichrome, and TUNEL staining to assess morphology, extracellular matrix status, and ablation status, respectively (FIG. 7B). FIG. 7B shows experimental results for urothelial resurfacing with IRE in healthy rats. FIG. 7B, subpanel (a) shows conformal nitinol electrode catheter for CAIR; subpanel (b) shows surgical approach used for WBUR with IRE in rats; subpanel (c) shows US image at 10 days post-IRE shows patent bladder; subpanel (d) shows H&E of whole mount bladder 4 hours post-IRE (inset left: no urothelium; inset right: blue stain (MT) for collagen and intact basement membrane); subpanel (e) shows 10 days post-IRE shows complete urothelialization (inset left: normal urothelium; inset right: blue stain (MT) for collagen shows no scarring; Asterisks indicate intact smooth muscle).

FIG. 7C shows experimental results for imaging of bladder volume post-IRE. Ultrasound-measurements demonstrated CAIR treatment does not appear to impact bladder volume or voiding in healthy or cancer bearing rats (FIG. 7C). In healthy rats, acute samples showed complete ablation of the urothelial layer on Day 1 followed by recovery to baseline status by Day 10 (FIG. 7D). In rats with bladder cancer, the use of CAIR appeared to result in rapid, predictable debulking of the urothelial layer within the entire bladder without injury to the underlying muscularis and submucosa (FIG. 7E-7G). CAIR treatment of rats with bladder cancer elicited regeneration of a healthy urothelial layer by Day 10 post-treatment.

FIG. 7E shows the gross appearance and complications following bladder IRE. FIG. 7E, subpanel (a) shows cross cross-sectional H&E-stained image of sham sham-treated bladder; subpanel (b) shows the cross cross-sectional H&E-stained image of IRE IRE-treated rat bladder. IRE IRE-treated bladder wall demonstrates loss or significant attenuation of the urothelial layer, hyperemia or bleeding in the bladder wall, and occasional loss of the muscularis; subpanel (c) shows a high magnification image of the urothelial layer demonstrating reduced thickness, equivalent to what would be seen in a healthy bladder.

FIG. 7F shows the penetration of IRE when performing whole bladder IRE, where H&E image shows the measurement approach where the bladder wall at various locations was evaluated for depth of urothelium, submucosa, and muscle in IRE and sham-treated animals.

FIG. 7G shows TUNEL staining for cell death following whole-bladder IRE, where subpanel (a) shows a cross section of IRE treated bladder showing diffuse brown staining indicative of cell death (boxes show locations for image); subpanel (b) shows Urothelial nuclei (arrows) stain positive (brown) for TUNEL confirming cell death; subpanel (c) shows diffuse staining is also observed in the muscularis of the bladder wall; subpanel (d) shows a cross section of sham treated rat bladder with little to no brown staining indicative of cell death; subpanel (e) shows higher magnification image of the urothelium.

Human-sized efficacy and safety of CAIR was tested in human-sized healthy swine bladders. Results showed that CAIR is scalable and reproducible at human size (FIG. 7H). 7H shows whole-bladder IRE in human-sized swine, where subpanel (a) shows a scaled basket electrode device employed in the testing, subpanel (b) shows an X-ray of device (arrow) in bladder (*), subpanel (c) shows an unfurled basket, subpanel (d) shows 24-hr CT showing intact bladder (arrow), subpanel (e) shows H&E of IRE treated bladder wall (arrows show denudation of the urothelial layer, muscularis is show with an asterisk).

FIG. 7I shows that the addition of calcium (see 750) during PEF application can increase cell death in low voltage regions, ensuring complete debulking or ablation even in regions between the electrodes that are not fully covered by a uniform electric field. The data shows that the addition of calcium can substantially increase cell death even in conditions when little to none is expected. The data also suggest that the exemplary system and method can promote a more pronounced ablation in gel models.

Example Device Design Characteristics. From the simulation, it can be observed that electrodes directly contacting tissue can cause a large increase in temperature with tissue directly adjacent to the electrodes due to Joule heating (FIG. 5G). The study developed a plastic cage and incorporated into the device as the outer most layer of the basket electrodes, acting as a spacer between the electrodes and the bladder lining to prevent direct contact of the electrode basket with the bladder wall (FIG. 6E). The offsetting of the electrodes from the bladder lining can reduce hot spots of energy delivery, thereby avoiding injury to healthy periphery tissue via thermal damage.

In combination with the basket electrode catheter, the study introduced a conductive fluid into the bladder to direct energy from the basket electrodes to the bladder wall. In addition to energy delivery, the conductive fluid provides a thermal heatsink from the Joule heating pulse effects, with the option for the fluid being chilled prior to entering the body for an increased heatsink. The conductive fluid can also normalize the electric field gradient specific to the urothelium layer, dispersing the energy delivery more evenly across the bladder wall than if the electrodes were directly contacting the bladder and were not surrounded by conductive medium.

A full bladder can contain up to 500 cc of fluid, and it was observed computationally that while increasing the conductivity of the fluid can improve the EFS gradient (FIG. 5F), it also increases current draw. A balloon or similar configuration mounted to the center of the catheter can fill most of the volume of the bladder lumen, limiting the amount of conductive fluid needed to provide complete contact between the electrodes and the bladder lining, thereby reducing the current drawn by the device during use.

Example Pulse Parameters. The study determined the pulse parameters associated with producing urothelial layer exfoliation, the pulse patterns can be grouped according to phase. Monophasic electric pulses can be achieved with a square wave, e.g., a voltage range of 100 V-1500V, pulse width of 50-150 μs, interpulse delay of 1 s, and with a minimum pulse number of 3-8 and a maximum of 900 pulses. Biphasic EP can be achieved with symmetric and asymmetric square pulses, a voltage range of 100 V-1500 V, pulse width of 5 ns-100 μs, 5 ns-1 s interpulse/interburst delay, and with a minimum pulse number of 3-8 a maximum pulse number of 900 pulses. These configurations, and other configurations described herein, may be employed.

Electroporation and Drug Delivery via Whole Bladder or Organs. The study determined that the exemplary pulsed electric energy system and method may be used to promote the delivery of drug, therapeutics, or other biological material into the bladder and other hollow or tubular organs. Pulsed electric field can temporarily modulate the barrier function of epithelial cells, allowing for the promotion of material delivery through the urothelial layer and deeper into the bladder wall.

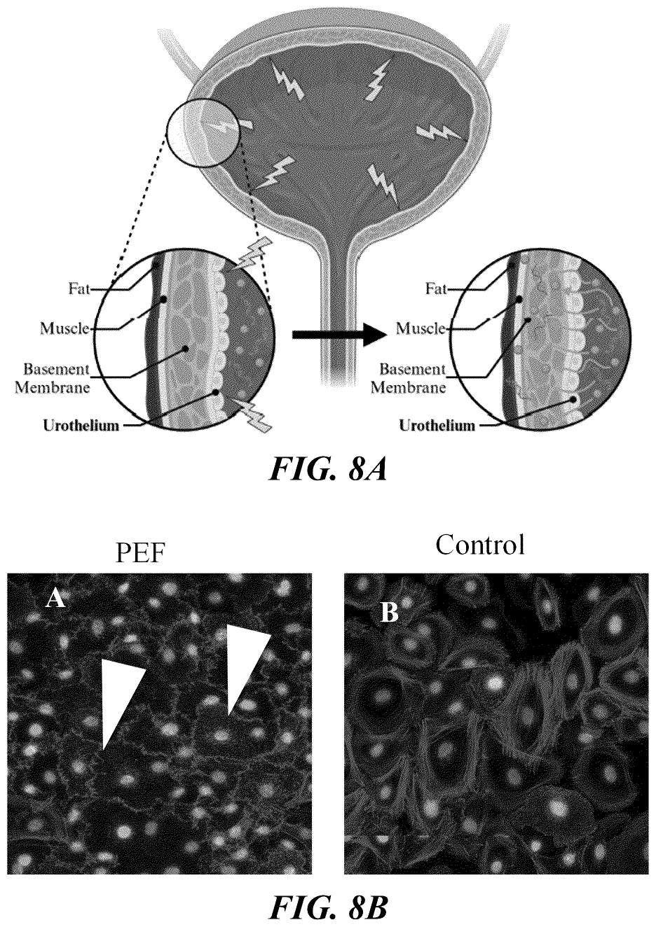

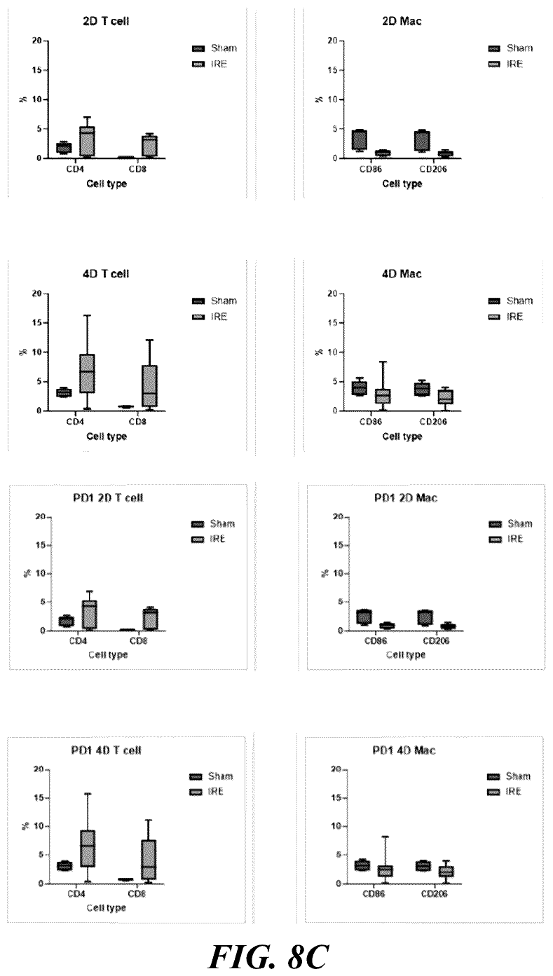

FIG. 8A shows PEF operation in the bladder can be used to temporarily disrupt the barrier function of epithelial cells for the promotion of material delivery.

FIG. 8C shows induction of an immunotherapy-friendly environment in the bladder. In the preliminary data of the study, the data show that a tumor microenvironment (TME) can be reprogrammed with IRE, where treatment shows an increase in activity of both macrophage and T-cell (PD1, PDL1, and CD3) populations.

FIG. 8B shows a Urothelial cell response to a pulsed electric field operation. FIG. 8B, subpanel (a) shows a pulsed electric field triggering actin remodeling that results in VE-Cadherin and ZO-1 loss from the cell interface, followed by internalization. Regions of intercellular gaps are shown with white arrows. The cellular changes appear to contribute to increased barrier permeability. The actin cytoskeleton is shown. FIG. 8B, subpanel (b) shows a comparison to data for untreated urothelial cells. Indeed, the selection of parameters for pulsed electric field can be used to spare a large number of T cells in the tumor microenvironment while killing the cancer cells. By killing only the cancer cells, the number of PD (e.g., macrophage and T-cell) can be increased.

Liquid Biosy. When cancer grows, it will naturally shed material into urine. However, the shedding is non-uniform and often biased toward more aggressively growing tumors that tend to shed more DNA, so the collection of the urine is often not an accurate representative of the cancer status in the bladder. Rather, conventional sampling provides an indication of the most aggressive cancer present in the bladder or the degree of aggressiveness of the cancer.

By applying pulsed electric energy to the whole bladder, e.g., in a single shot, as described herein, the entire bladder wall can be sampled as a biopsy having RNA and DNA that can be employed for sequencing. The result is an unbiased sample that provides a more comprehensive genomic snapshot or analysis of the bladder.

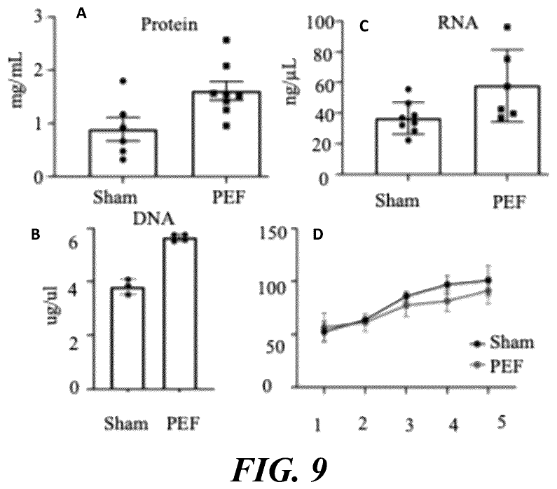

FIG. 9 shows in vitro image data using PEF for liquid biopsy generation. The PEF is delivered in a solid MB49 tumor. In FIG. 9, EFAB samples are observed to have greater protein (subpanel (a), RNA (subpanel (b), and DNA (subpanel (c)) content than sham. FIG. 9, subpanel (d) shows results of mice undergoing EFAB exhibiting tumor growth similar to sham. The data shows that the application of the pulsed electric field application can cause urothelial cells that are normally connected tightly to each other to lose their barrier function and open up, allowing for the diffusion of material through them into the bladder wall more easily.

### DISCUSSION

The molecular and genetic characteristics of bladder cancer render it likely to develop resistance and escape therapies that targets specific or even multiple biological pathways [24], [28], [38], [39]. The surgical removal of all bladder urothelium during RC provides durable cancer control but its use is restricted to high-risk patients with muscle-invasive Bladder cancer or after failure of intravesical therapy [33], [40]. Prior research demonstrates that ablation can rapidly eliminate all urothelium from the bladder, similar to RC [41], [42], but with the advantage of being a minimally invasive, bladder-sparing technique, rendering it an attractive option for patients who are not candidates for RC. Unlike focal tumor ablation, where complete eradication of all malignant cells is mandatory, debulking of dysplastic urothelium by itself may provide tumor risk reduction.

Urothelial cells have a tremendous capacity to repopulate and regenerate large defects in the bladder [43], [44] as evidenced by prior studies showing complete recovery of the urothelial layer following whole-bladder laser ablation in a canine model⁴¹. The upper urinary tract served as the reservoir of urothelial cells for reseeding and regenerating the bladder urothelial layer in that study, and similar mechanisms have been confirmed in other studies as well [43], [44]. Interestingly, the urothelium of the upper urinary tract remains healthy or cancer-free in both the chemically induced Bladder cancer model in rats used in our proposed experiments [45], as well as in the majority of patients with Bladder cancer [2], [7]. Based on this body of literature, we rationalize that rapid resurfacing of bladder urothelium with IRE will be followed by regeneration of a disease-free layer seeded by cells from the upper urinary tract (FIG. 7B).

Example Computing System. The exemplary system and method may be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUS), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well-known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

### CONCLUSION

The construction and arrangement of the systems and methods as shown in the various implementations are illustrative only. Although only a few implementations have been described in detail in this disclosure, many modifications are possible (e.g., variations in sizes, dimensions, structures, shapes, proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementation of the present disclosure may be implemented using existing computer processors, or by a computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

Each and every feature described herein, and each and every combination of two or more of such features, is included within the scope of the present invention, provided that the features included in such a combination are not mutually inconsistent.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.
[1] Cancer Statistics 2020. Accessed Oct. 3, 2020. https://www.cancer.org/content/dam/cancer-org/research/cancer-facts-and-statistics/annual-cancer-facts-and-figures/2020/cancer-facts-and-figures-2020.pdf
[2] Kirkali Z, Chan T, Manoharan M, Algaba F, Busch C, Cheng L, Kiemeney L, Kriegmair M, Montironi R, Murphy W M, Sesterhenn I A, Tachibana M, Weider J. Bladder cancer: Epidemiology, staging and grading, and diagnosis. In: Urology. Vol 66. Urology; 2005:4-34. doi:10.1016/j.urology.2005.07.062
[3] Mertens L S, Neuzillet Y, Horenblas S, Van Rhijn B W G. Landmarks in non-muscle-invasive bladder cancer. Nat Rev Urol. 2014; 11 (8): 476-480. doi: 10.1038/nrurol.2014.130
[4] Burger M, Catto J W F, Dalbagni G, Grossman H B, Herr H, Karakiewicz P, Kassouf W, Kiemeney L A, La Vecchia C, Shariat S, Lotan Y. Epidemiology and risk factors of urothelial bladder cancer. Eur Urol. 2013; 63 (2): 234-241. doi: 10.1016/j.eururo.2012.07.033
[5] Bladder Cancer: Non-Muscle Invasive Guideline-American Urological Association. Accessed Jan. 12, 2021. https://www.auanet.org/guidelines/bladder-cancer-non-muscle-invasive-guideline
[6] Sylvester R J, Van Der Meijden A P M, Oosterlinck W, Witjes J A, Bouffioux C, Denis L, Newling D W W, Kurth K. Predicting recurrence and progression in individual patients with stage Ta Tl bladder cancer using EORTC risk tables: A combined analysis of 2596 patients from seven EORTC trials. Eur Urol. 2006; 49 (3): 466-477. doi: 10.1016/j.eururo.2005.12.031
[7] Leblanc B, Duclos A J, Bénard F, Côté J, Valiquette L, Paquin J M, Mauffette F, Faucher R, Perreault J P. Long-term followup of initial Ta grade 1 transitional cell carcinoma of the bladder. J Urol. 1999; 162 (6): 1946-1950. doi: 10.1016/S0022-5347 (05) 68075-5
[8] Sanchez A, Wszolek M F, Niemierko A, Clayman R H, Drumm M, Rodríguez D, Feldman A S, Dahl D M, Heney N M, Shipley W U, Zietman A L, Efstathiou J A. Incidence, Clinicopathological Risk Factors, Management and Outcomes of Nonmuscle Invasive Recurrence after Complete Response to Trimodality Therapy for Muscle Invasive Bladder Cancer. J Urol. 2018; 199 (2): 407-415. doi: 10.1016/j.juro.2017.08.106
[9] Millán-Rodríguez F, Chéchile-Toniolo G, Salvador-Bayarri J, Palou J, Algaba F, Vicente-Rodríguez J. Primary superficial bladder cancer risk groups according to progression, mortality and recurrence. J Urol. 2000; 164 (3 I): 680-684. doi: 10.1016/S0022-5347 (05) 67280-1
[10] Stein J P, Lieskovsky G, Cote R, Groshen S, Feng A C, Boyd S, Skinner E, Bochner B, Thangathurai D, Mikhail M, Raghavan D, Skinner D G. Radical cystectomy in the treatment of invasive bladder cancer: Long-term results in 1,054 patients. J Clin Oncol. 2001; 19 (3): 666-675. doi: 10.1200/JCO.2001.19.3.666
[11] Jäger W, Thomas C, Haag S, Hampel C, Salzer A, Thüroff J W, Wiesner C. Early vs delayed radical cystectomy for “high-risk” carcinoma not invading bladder muscle: Delay of cystectomy reduces cancer-specific survival. BJU Int. 2011; 108 (8 B). doi: 10.1111/j.1464-410X.2010.09980.x
[12] Wan J C M. Survival Outcomes of Early versus Deferred Cystectomy for High-Grade Non-Muscle-Invasive Bladder Cancer: A Systematic Review. Curr Urol. 2020; 14 (2): 66-73. doi: 10.1159/000499257
[13] Shabsigh A, Korets R, Vora K C, Brooks C M, Cronin A M, Savage C, Raj G, Bochner B H, Dalbagni G, Herr H W, Donat S M. Defining Early Morbidity of Radical Cystectomy for Patients with Bladder Cancer Using a Standardized Reporting Methodology. Eur Urol. 2009; 55 (1): 164-176. doi: 10.1016/j.eururo.2008.07.031
[14] Knorr J M, Ericson K J, Zhang J H, Murthy P, Nowacki A S, Munoz-Lopez C, Thomas L J, Haber G-P, Lee B. Comparison of Major Complications at 30 and 90 Days Following Radical Cystectomy. Urology. Published online September 2020. doi: 10.1016/j.urology.2020.08.038
[15] Williams S B, Cumberbatch M G K, Kamat A M, Jubber I, Kerr P S, McGrath J S, Djaladat H, Collins J W, Packiam V T, Steinberg G D, Lee E, Kassouf W, Black P C, Cerantola Y, Catto J W F, Daneshmand S. Reporting Radical Cystectomy Outcomes Following Implementation of Enhanced Recovery After Surgery Protocols: A Systematic Review and Individual Patient Data Meta-analysis. Eur Urol. 2020; 0 (0). doi: 10.1016/j.eururo.2020.06.039
[16] Cerantola Y, Valerio M, Persson B, Jichlinski P, Ljungqvist O, Hubner M, Kassouf W, Muller S, Baldini G, Carli F, Naesheimh T, Ytrebo L, Revhaug A, Lassen K, Knutsen T, Aarsether E, Wiklund P, Patel H R H. Guidelines for perioperative care after radical cystectomy for bladder cancer: Enhanced recovery after surgery (ERAS®) society recommendations. Clin Nutr. 2013; 32 (6): 879-887. doi: 10.1016/j.clnu.2013.09.014
[17] Check D K, Leo M C, Banegas M P, Bulkley J E, Danforth K N, Gilbert S M, Kwan M L, Rosetti M O, McMullen C K. Decision Regret Related to Urinary Diversion Choice among Patients Treated with Cystectomy. J Urol. 2020; 203 (1): 159-163. doi: 10.1097/JU.0000000000000512
[18] Gupta N, Rasmussen SEVP, Haney N, Smith A, Pierorazio P M, Johnson M H, Hoffman-Censits J, Bivalacqua T J. Understanding psychosocial and sexual health concerns among women with bladder cancer undergoing radical cystectomy. Urology. Published online August 2020. doi: 10.1016/j.urology.2020.08.018
[19] Sidransky D, Frost P, Von Eschenbach A, Oyasu R, Preisinger A C, Vogelstein B. Clonal Origin of Bladder Cancer. N Engl J Med. 1992; 326 (11): 737-740. doi: 10.1056/nejm199203123261104
[20] Strandgaard T, Nordentoft I, Lamy P, Christensen E, Thomsen M B H, Jensen J B, Dyrskjøt L. Mutational analysis of field cancerization in bladder cancer. Bl Cancer. 2020; 6 (3): 253-264. doi: 10.3233/BLC-200282
[21] Messing E M, Tangen C M, Lerner S P, et al. Effect of intravesical instillation of gemcitabine vs saline immediately following resection of suspected low-grade non-muscle-invasive bladder cancer on tumor recurrence SWOG S0337 randomized clinical trial. JAMA-J Am Med Assoc. 2018; 319 (18): 1880-1888. doi: 10.1001/jama.2018.4657
[22] Perlis N, Zlotta A R, Beyene J, Finelli A, Fleshner N E, Kulkarni G S. Immediate post-transurethral resection of bladder tumor intravesical chemotherapy prevents non-muscle-invasive bladder cancer recurrences: An updated meta-analysis on 2548 patients and quality-of-evidence review. Eur Urol. 2013; 64 (3): 421-430. doi: 10.1016/j.eururo.2013.06.009
[23] Steinberg R L, Thomas L J, Brooks N, Mott S L, Vitale A, Crump T, Rao M Y, Daniels M J, Wang J, Nagaraju S, DeWolf W C, Lamm D L, Kates M, Hyndman M E, Kamat A M, Bivalacqua T J, Nepple K G, O'Donnell M A. Multi-Institution Evaluation of Sequential Gemcitabine and Docetaxel as Rescue Therapy for Nonmuscle Invasive Bladder Cancer. J Urol. 2020; 203 (5): 902-909. doi: 10.1097/JU.0000000000000688
[24] Taber A, Christensen E, Lamy P, Nordentoft I, Prip F, Lindskrog S V, Birkenkamp-Demtröder K, Okholm T L H, Knudsen M, Pedersen J S, Steiniche T, Agerbæk M, Jensen J B, Dyrskjøt L. Molecular correlates of cisplatin-based chemotherapy response in muscle invasive bladder cancer by integrated multi-omics analysis. Nat Commun. 2020; 11 (1): 1-15. doi: 10.1038/s41467-020-18640-0
[25] Cookson M S, Chang S S, Lihou C, li T, Harper S Q, Lang Z, Tutrone R F. Use of intravesical valrubicin in clinical practice for treatment of nonmuscle-invasive bladder cancer, including carcinoma in situ of the bladder. Ther Adv Urol. 2014; 6 (5): 181-191. doi: 10.1177/1756287214541798
[26] Robins D J, Sui W, Matulay J T, Ghandour R, Anderson C B, DeCastro G J, McKiernan J M. Long-term Survival Outcomes With Intravesical Nanoparticle Albumin-bound Paclitaxel for Recurrent Non-muscle-invasive Bladder Cancer After Previous Bacillus Calmette-Guérin Therapy. Urology. 2017; 103:149-153. doi: 10.1016/j.urology.2017.01.018
[27] Wang C, Li A, Yang S, Qiao R, Zhu X, Zhang J. CXCL5 promotes mitomycin C resistance in non-muscle invasive bladder cancer by activating EMT and NF-κB pathway. Biochem Biophys Res Commun. 2018; 498 (4): 862-868. doi: 10.1016/j.bbrc.2018.03.071
[28] Vallo S, Michaelis M, Rothweiler F, Bartsch G, Gust K M, Limbart D M, Rödel F, Wezel F, Haferkamp A, Cinatl J. Drug-resistant urothelial canc cell lines display diverse sensitivity profiles to potential second-line therapeutics. Transl Oncol. 2015; 8 (3): 210-216. doi: 10.1016/j.tranon.2015.04.002
[29] Arends T J H, Van Der Heijden A G, Witjes J A. Combined chemohyperthermia: 10-year single center experience in 160 patients with nonmuscle invasive bladder cancer. J Urol. 2014; 192 (3): 708-713. doi: 10.1016/j.juro.2014.03.101
[30] Juvet T, Mari A, Lajkosz K, Wallis C J, Kuk C, Erlich A, Krimus L, Fleshner N E, Kulkarni G S, Zlotta A R. Sequential administration of Bacillus Calmette-Guerin (BCG) and Electromotive Drug Administration (EMDA) of mitomycin C (MMC) for the treatment of high-grade nonmuscle invasive bladder cancer after BCG failure. Urol Oncol Semin Orig Investig. 2020; 38 (11): 850.e9-850.e15. doi: 10.1016/j.urolonc.2020.06.031
[31] Kamat A M, Hahn N M, Efstathiou J A, Lerner S P, Malmström PU, Choi W, Guo C C, Lotan Y, Kassouf W. Bladder cancer. Lancet. 2016; 388 (10061): 2796-2810. doi: 10.1016/S0140-6736 (16) 30512-8
[32] Krajewski W, Moschini M, Chorbińska J, et al. Delaying BCG immunotherapy onset after transurethral resection of non-muscle-invasive bladder cancer is associated with adverse survival outcomes. World J Urol. Published online Nov. 23, 2020:1-8. doi: 10.1007/s00345-020-03522-3
[33] Kamat A M, Colombel M, Sundi D, Lamm D, Boehle A, Brausi M, Buckley R, Persad R, Palou J, Soloway M, Witjes J A. BCG-unresponsive non-muscle-invasive bladder cancer: Recommendations from the IBCG. Nat Rev Urol. 2017; 14 (4): 244-255. doi: 10.1038/nrurol.2017.16
[34] Li R, Lerner S P, Kamat A M. The Who, What, When, Where, and Why of Bacillus Calmette-Guérin-unresponsive Bladder Cancer. Eur Urol. Published online Jan. 14, 2021. doi: 10.1016/j.eururo.2021.01.001
[35] Joudi F N, Smith B J, O'Donnell M A. Final results from a national multicenter phase II trial of combination bacillus Calmette-Guérin plus interferon α-2B for reducing recurrence of superficial bladder cancer {star, open}. Urol Oncol Semin Orig Investig. 2006; 24 (4 SPEC. ISS.): 344-348. doi: 10.1016/j.urolonc.2005.11.026
[36] Balar A V, Kulkarni G S, Uchio E M, Boormans J, Mourey L, Krieger L E M, Singer E A, Bajorin D F, Kamat A M, Grivas P, Seo H K, Nishiyama H, Konety B R, Nam K, Kapadia E, Frenkl T L, De Wit R. Keynote 057: Phase I I trial of Pembrolizumab (pembro) for patients (pts) with high-risk (HR) nonmuscle invasive bladder cancer (NMIBC) unresponsive to bacillus calmette-guérin (BCG). J Clin Oncol. 2019; 37 (7_suppl): 350-350. doi: 10.1200/jco.2019.37.7_suppl.350
[37] Boorjian S A, Alemozaffar M, Konety B R, et al. Intravesical nadofaragene firadenovec gene therapy for BCG-unresponsive non-muscle-invasive bladder cancer: a single-arm, open-label, repeat-dose clinical trial. Lancet Oncol. 2021; 22 (1): 107-117. doi: 10.1016/S1470-2045 (20) 30540-4
[38] Meeks J J, Al-Ahmadie H, Faltas B M, Taylor J A, Flaig T W, DeGraff D J, Christensen E, Woolbright B L, McConkey D J, Dyrskjøt L. Genomic heterogeneity in bladder cancer: challenges and possible solutions to improve outcomes. Nat Rev Urol. 2020; 17 (5): 259-270. doi: 10.1038/s41585-020-0304-1
[39] Hafner C, Knuechel R, Stoehr R, Hartmann A. Clonality of multifocal urothelial carcinomas: 10 Years of molecular genetic studies. Int J Cancer. 2002; 101 (1): 1-6. doi: 10.1002/ijc.10544
[40] Catto J W F, Gordon K, Collinson M, Poad H, Twiddy M, Johnson M, Jain S, Chahal R, Simms M, Dooldeniya M, Bell R, Koenig P, Conroy S, Goodwin L, Noon A P, Croft J, Brown J M. Radical Cystectomy Against Intravesical BCG for High-Risk High-Grade Nonmuscle Invasive Bladder Cancer: Results From the Randomized Controlled BRAVO-Feasibility Study. J Clin Oncol. Published online Dec. 17, 2020: JCO.20.01665. doi: 10.1200/jco.20.01665
[41] Wishnow K I, Johnson D E, Grignon D, Ayala A G, Cromeens D, von Eschenbach A C. Denudation of the entire mucosa of the canine urinary bladder using the neodymium: YAG laser with the MTR 1.5 contact probe. Lasers Surg Med. 1988; 8 (6): 589-595. doi: 10.1002/lsm.1900080609
[42] Z X, K B, J T, RB M. Whole bladder photodynamic therapy for orthotopic superficial bladder cancer in rats: a study of intravenous and intravesical administration of photosensitizers. J Urol. 2003; 169 (1). doi: 10.1097/01.JU.0000039350.39402.F0
[43] Yamany T, Van Batavia J, Mendelsohn C. Formation and regeneration of the urothelium. Curr Opin Organ Transplant. 2014; 19 (3): 323-330. doi: 10.1097/MOT.0000000000000084
[44] Veranič P, Erman A, Kerec-Kos M, Bogataj M, Mrhar A, Jezernik K. Rapid differentiation of superficial urothelial cells after chitosan-induced desquamation. Histochem Cell Biol. 2009; 131 (1): 129-139. doi: 10.1007/s00418-008-0492-x
[45] Mori S, Hosono M, Machino S, Nakai S, Makino S, Nakao H, Takeuchi Y, Takeda R, Murai T, Fukushima S. Induction of the renal pelvic and ureteral carcinomas by N-butyl-N-(4-hydroxybutyl) nitrosamine in SD/cShi rats with spontaneous hydronephrosis. Toxicol Pathol. 1994; 22 (4): 373-380. doi: 10.1177/019262339402200403

1. A system comprising:
an expandable electrode catheter comprising:
a tubular member having a first end and a second end; and
a plurality of electrodes comprising at least 4 electrodes including a first electrode and a second electrode, the plurality of electrodes housed in the first end of the tubular member and configured (i) to be stowed within the tubular member in a first configuration and (ii) to extend out of the tubular member, via movement of the tubular member or movement of the plurality of electrodes, to be in a second configuration,
wherein during deployment the first electrode extends in a first angular direction and second electrode extends in a second angular direction opposite to the first angular direction so each come into proximity or contact with a surface tissue of a hollow or tubular organ to fill a shape of the hollow or tubular organ,
wherein each of the plurality of electrodes is configured to uniformly directly or indirectly emit a plurality of electrical pulses in a liquid medium (e.g., while in contact with the tissue or only through the liquid medium) to penetrate at least a pre-defined depth into the surface tissue as a pulsed electric field application to induce a tissue response (e.g., irreversible electroporation and subsequent exfoliation, electroporation, pulsed field ablation, cell stimulation, and other non-thermal ablation) of at least the surface tissue of the hollow or tubular organ.

2. The system of claim 1, wherein the flexible tubular member comprises a channel member that extends, at least, between the first end and the second end of the tubular member for introduction of a liquid, as a portion of the liquid medium, to be dispensed during the pulsed electric field application by the expandable electrode catheter (e.g., wherein the liquid and liquid medium can limit the focal or localized ablation of the surface tissue of the hollow or tubular organ or the off-target destruction of other biological material in the hollow or tubular organ, such as blood or other bodily fluids).

3. The system of claim 1, wherein the expandable electrode catheter further comprises:
an expandable sheath (e.g. balloon) comprising a non-conductive material, the expandable sheath housed in the first end of the tubular member and configured (i) to be stowed within the tubular member in a stowed configuration and (ii) to extend out of the tubular member to define a sheath volume defined within the plurality of electrodes, wherein the sheath volume when deployed reduces a volume in a space in the hollow or tubular organ for delivery of electrical pulse in the liquid medium to the hollow or tubular organ.

4. The system of claim 1, wherein the expandable electrode catheter comprises:
a tubular sheath forming in part the tubular member, the tubular sheath having a first end and a second end; and
a shaft housed within the tubular sheath and configured to be displaced (e.g., slidable) within the sheath, the shaft having a first end and a second end, wherein the second end of the shaft is configured to controllably move within the second end of the sheath to induce deployment of the plurality of electrodes.

5. The system of claim 1, wherein the expandable electrode catheter comprises:
a tubular sheath forming in part the tubular member, the tubular sheath having a first end and a second end; and
a shaft housed within the tubular sheath, a portion of the tubular sheath being configured to be displaced (e.g., slidable) with respect to the shaft, the shaft having a first end and a second end, wherein the second end of the shaft is configured to controllably move to the second end of the sheath to induce deployment of the plurality of electrodes.

6. The system of claim 1, wherein the plurality of electrodes form a basket configured to conform to a shape of the hollow or tubular organ.

7. The system of claim 1, wherein the plurality of electrodes are between 4 and 100 electrode members, and wherein the plurality of electrodes are configured to flexibly extend to contact and oppose a lumen wall as the surface tissue without losing contact.

8. The system of claim 1, wherein the electrode members are configured to extend about 1 mm-75 mm from the tubular member to have at least one electrode member positionable 0 to 5 mm from a lumen wall as the tissue surface.

9. The system of claim 1, wherein the expandable electrode catheter further comprises an expandable non-conductive envelop, the expandable non-conductive envelope having a plurality of expandable members housed at the first end of the tubular member and configured (i) to be stowed to define an outer surface the tubular member in a stowed configuration and (ii) to extend or expand from the tubular member to define an external envelope volume, wherein the plurality of electrodes expands in the second configuration to be defined within the external envelope volume, wherein the expandable non-conductive envelope defines a pre-defined gap or distance between the plurality of electrodes and the surface tissue.

10. The system of claim 1, wherein the expandable electrode catheter is coupled to a handle, the handle being manipulatable to move the plurality of electrodes within the hollow or tubular organ.

11. The system of claim 1 further comprising:
an electrical instrument configured to deliver the plurality of electrical pulses to the plurality of electrodes.

12. The system of claim 11, wherein the electrical instrument is configured, via a control program for a pulsed electric field application (e.g., electroporation, irreversible electroporation, pulsed field ablation or targeted cell stimulation), to generate a monophasic electro pulse having a square wave.

13. The system of claim 11, wherein the electrical instrument is configured, via a control program for irreversible electroporation, to output a voltage range of 50 V-1500V (e.g., 50 Vₚₑₐₖ-1500 Vₚₑₐₖ), and pulse width of 10 μs-1000 μs (e.g., 50-150 μs).

14. The system of claim 11, wherein the electrical instrument is configured, via a control program for irreversible electroporation, to generate electropulses having a selectable interpulse delay (e.g., triggerable via an external biosignal, e.g., muscle contraction, nerve excitation, or cardiac rhythm).

15. having a minimum of 3-8 electropulses and a maximum of 10,000 electropulses).

16. The system of claim 11, wherein the electrical instrument is configured, via a control program for irreversible electroporation, to generate biphasic electropulses comprising symmetric or asymmetric square waves.

17. The system of claim 16, wherein the electrical instrument is configured, via the control program, to generate the biphasic electropulses at a voltage range of 100 V-1500 V (e.g., 100Vₚₚ-1500 Vₚₚ), and pulse width of 5 ns-50 μs.

18. The system of claim 16, wherein the electrical instrument is configured, via the control program, to generate electropulses comprising a 5 ns-1 s interpulse delay.

19. The system of claim 16, wherein the electrical instrument is configured, via the control program, to deliver a pre-defined number of electropulses (e.g. having a minimum of 3-8 electropulses and a maximum of 10,000 electropulses).

20. The system of claim 1, further comprising:
a liquid introducer device, the liquid introducer device being configured, via control, to introduce the liquid into the hollow or tubular organ during the pulsed electric field application and maintain the conductivity ratio in a pre-defined range (e.g., a conductivity ratio between 1 and 10 between the liquid medium in the hollow or tubular organ and the surface tissue of the hollow or tubular organ when the plurality of electrodes are intended to contact the surface tissue during the delivery of the electropulses) (e.g., a conductivity ratio between 0.1 and 0.5 between the liquid medium in the hollow or tubular organ and the surface tissue of the hollow or tubular organ when the plurality of electrodes are intended to not contact the surface tissue during the delivery of the electropulses (e.g., when plastic spacer cage is employed)).

21. The system of claim 1 further comprising:
a recirculation loop instrument, the recirculation loop instrument being coupled to the expandable electrode catheter or a delivery device to actively recirculate the liquid and portion of the liquid medium during the pulsed electric field application (e.g., to reduce localized tissue heating).

22. An expandable electrode catheter comprising:
a tubular member having a first end and a second end; and
a plurality of electrodes comprising at least 4 electrodes including a first electrode and a second electrode, the plurality of electrodes housed in the first end of the tubular member and configured (i) to be stowed within the tubular member in a first configuration and (ii) to extend out of the tubular member, via movement of the tubular member or movement of the plurality of electrodes, to be in a second configuration, wherein during deployment the first electrode extends in a first angular direction and second electrode extends in a second angular direction opposite to the first angular direction so each come into proximity or contact with a surface tissue of a hollow or tubular organ to fill a shape of the hollow or tubular organ,
wherein each of the plurality of electrodes is configured to uniformly emit a plurality of electrical pulses in a liquid medium to penetrate at least a pre-defined depth into the surface tissue to induce irreversible electroporation and subsequent exfoliation of the surface tissue of the hollow or tubular organ.

23.-42. (canceled)
